(19) United States
(12) Patent Application Publication
(10) Pub. No.: US 2025/0375222 A1
(43) Pub. Date: Dec. 11, 2025

(54) SPINAL FIXATION SYSTEM WITH RECEIVER AND INSERT SUB-ASSEMBLIES FOR CONNECTING WITH BI-SPHERIC UNIVERSAL SHANK HEADS

(71) Applicant: Roger P. Jackson, Prairie Village, KS (US)

(72) Inventors: Roger P. Jackson, Prairie Village, KS (US); James L. Surber, Kansas City, KS (US); Nathaniel D. Ginzton, Boise, ID (US)

(21) Appl. No.: 19/234,110

(22) Filed: Jun. 10, 2025

Related U.S. Application Data
(60) Provisional application No. 63/658,082, filed on Jun. 10, 2024.

(51) Int. Cl. A61B 17/70; A61B 17/00; A61B 17/86

(52) U.S. Cl. CPC A61B 17/7035; A61B 17/8605; A61B 17/8625; A61B 17/864; A61B 2017/00477

(57) ABSTRACT

A spinal fixation system includes a plurality of receiver sub-assemblies and bone anchors with shank heads, with each receiver sub-assembly including a receiver having a channel for receiving a rod and a base defining a cavity in communication with the channel to define a central bore. Each receiver sub-assembly also includes a retaining structure positionable in the cavity for frictionally engaging and articulating with an upper partial spherical surface of a shank head. Each receiver sub-assembly further includes an insert sub-assembly positionable within the central bore above the retaining structure, with the insert sub-assembly including a support collar with outer surfaces for engaging interior surfaces of the central bore, a load saddle supported by the support collar with an upper surface for engaging the elongate rod, and a clamp positioner also supported by the support collar with curvate inner surfaces for engaging the retaining structure before and after it frictionally engages the shank head.

### CROSS-REFERENCE TO RELATED APPLICATIONS

This application claims the benefit of U.S. Provisional Application No. 63/658,082, filed Jun. 10, 2024, which is incorporated by reference in its entirety herein and for all purposes.

### FIELD

The present disclosure relates generally to modular spinal implant assemblies utilizing universal shank heads that are configured for connection with a collection or array of pivoting and non-pivoting but axially rotatable (e.g., monoaxial) receiver sub-assemblies having different functionalities, and their use in surgery involving vertebral body stabilizations with spinal fixation systems.

### BACKGROUND

Spinal implants in general, and bone anchors or screws in particular, are used in many types of spinal surgery in order to secure various implants to vertebrae along the spinal column for the purposes of treating spinal disorders, such as degenerative conditions and deformities, and also for stabilizing and/or adjusting spinal alignment. A common mechanism for providing vertebral support is to implant the bone screws into certain bones which then, in turn, support a longitudinal structure such as an elongate rod, or are supported by such a rod. Although both closed-ended and open-ended spinal implants, such as bone screws and hooks, are known, the open-ended spinal implants can be particularly well suited for connections to rods and connector arms because such rods or arms do not need to be passed through a closed bore, but rather can be laid or urged into an open channel within the head or receiver of such a screw, hook, or connector. For example, open-ended bone screws generally comprise an anchor portion, such as a threaded shank, connected to a head or receiver having a pair of upwardly-projecting branches or arms which form a yoke that defines a slot or channel configured to receive the rod. The slot or channel could have different shapes, such as, a U-shape or a square shape. Moreover, the threaded shanks of the bone screws can also be replaced with hooks or other types of bone anchors or connectors to form a variety of different types of spinal implants, also having open ends for receiving rods or portions of other structures, and wherein such implants can facilitate surgical techniques performed with different spinal fixation systems.

Early bone screws or anchors used in spinal surgery generally had a yoke-shaped ‘head’ that was integrally formed or “fixed” with the threaded shank, and therefore immovable. Because the fixed head could not be moved relative to the shank, these fixed bone screws needed to be favorably positioned in the spine; otherwise, the elongate rod would need to be bent in order for it to be placed within the rod-receiving channels of a linear series of adjacent bone screws, due to their alignment. Given the highly curved shape of the spines of some patients, however, this is sometimes very difficult or impossible to do. Therefore, polyaxial (i.e., multiplanar), uni-planar (i.e., monoplanar), and/or translatable pivotal bone screws or bone anchor assemblies, were developed and are now commonly preferred. Open-ended polyaxial bone screw assemblies typically allow for pivoting and rotation of the connected but completely separate yoke-shaped receiver or receiver sub-assembly about an enlarged spherical ‘head’ or upper capture portion of the threaded shank or bone anchor in one or more planes, until a desired rotational and pivotal position of the receiver is achieved relative to the shank. This can be accomplished by manipulating the position of the receiver relative to the shank during a final stage of a medical procedure when the elongate rod or other longitudinal connecting member is inserted into the receiver or receiver sub-assembly, followed by a locking set screw, a plug, a closure, or other type of hard locking mechanism known in the art.

It is understood that spinal fixation systems generally include a variety of components that require some assembly, such as the various types of bone anchors, the rods or connector arms, and the closures or plugs with the receivers or receiver sub-assemblies, with each component having specific features with respect to structure and function. Moreover, the receiver sub-assemblies can further include components in addition to the receiver itself, such as pressure inserts, wave washers, separate retainers, and other components of different types that are operable to connect these receiver sub-assemblies with the heads of the bone anchors. The pressure inserts, rings, retainers, and other components can be pre-assembled together within the receivers to form the receiver sub-assemblies that are ready for further assemblage with the bone anchors, and eventually with the rods or connector arms and the closures or plugs.

Some designs provide for the threaded shanks or other types of bone anchors to be bottom loaded into the receiver sub-assemblies. With bottom loaded bone anchor assemblies, for example, some designs known in the art require a retaining component (e.g., the collet portion of an insert or a separate retainer) to hold the shank within the receiver, with the receiver having a bottom opening large enough to allow for the head or upper capture portion of the threaded shank or bone anchor to be uploaded into the central bore or cavity of the receiver. Other types of bottom loaded bone anchor assemblies do not include the retaining component, however, and instead include a receiver having a lower portion with a bottom opening that is configured to directly threadably mate with the head or upper capture portion of the shank that can be configured as a threaded spherical head to provide for polyaxial or multiplanar motion.

Further to the above, bottom loaded bone anchor assemblies can also be fully assembled by the spinal company or distributor before being shipped to a hospital, so as to help with inventory management, or can be shipped as a modular array of multiple separate and different shanks and a fewer number of pre-assembled receiver sub-assemblies that can then be fully assembled, for example, at the hospital or surgical center during a surgery, thereby saving costs. Additionally, the modular spinal implants can be fully assembled at the hospital either before insertion into the patient, or after the threaded shank or bone anchor has been inserted into the patient, either by a surgeon, with or without robotic assistance, or also directly by a robot. The different techniques or approaches for the insertion and assembly of the modular parts of the bone anchor assemblies can be described as ex-vivo and in-vivo, respectively.

### SUMMARY

The present disclosure is generally directed to modular spinal fixation systems with bone anchors comprising a certain type of common or universal shank head configured to connect with a wide array of receiver sub-assemblies having different functionalities to form pivotal and non-pivotal bone anchor assemblies with different capabilities. To that purpose, one embodiment of the present disclosure comprises a spinal fixation system for securing an elongate rod to a spine of a patient.

The spinal fixation system includes a plurality of bone anchors, with each bone anchor having a longitudinal axis, a shank head at a proximal end devoid of outer parallel planar side surfaces, an anchor portion opposite the shank head configured for fixation to the bone, and a neck portion extending between the shank head and the anchor portion. Each shank head includes an upper partial spherical portion comprising an upper spherical surface having a first diameter extending downward from an upper end, out and around the hemisphere plane of the upper spherical surface, to a circular inner edge of upward-facing shelf surface of a lower shelf or ledge structure that is spaced below the hemisphere plane, and a lower partial spherical portion comprising a lower spherical surface having a second diameter that is greater than the first diameter and which extends downward from the circular outer edge of the upward-facing shelf surface toward the neck portion that connects the shank head to the anchor portion. In one aspect the upward-facing shelf surface is an annular planar surface extending perpendicular to the longitudinal axis of the bone anchor.

The spinal fixation system also includes an array of receiver sub-assemblies, with each receiver sub-assembly including a receiver with a base portion that defines a lower section of a central bore centered around a vertical centerline axis and communicating with a bottom of the receiver through a bottom opening, and an upper portion having a channel configured to receive the elongate rod describe above. The central bore includes a seating surface adjacent or proximate the bottom opening, and extends upward through the channel to a top of the receiver. Each receiver sub-assembly also includes one of a multiplanar pivoting retaining structure (also known as a cap retainer), a monoplanar pivoting retaining structure or cap retainer, or a non-pivoting or monoaxial retaining structure or cap retainer positioned therein and configured to slidably engage the seating surface after capturing the upper partial spherical portion of a shank head upon its uploading through the bottom opening of the receiver.

Each receiver sub-assembly further includes an insert sub-assembly positionable within the central bore above the retaining structure. The insert sub-assembly generally includes a load saddle configured to engage the elongate rod and a clamp positioner configured to engage the retaining structure both before and after the uploading of the bi-spheric shank head into the receiver sub-assembly. The insert sub-assemblies can also include additional components, such as a central collar, a wave washer, and a crown element, with all of the components of the insert sub-assemblies working together to establish a non-floppy, pre-lock friction fit between the bone anchor and the receiver upon the downward deployment of the insert sub-assembly with tooling.

After the shank head of the bone anchor is captured by the retaining structure or cap retainer of one of the retainer sub-assemblies, ex-vivo or in-vivo, and to form a bone anchor assembly, the bone anchor is further configured to have frictional axial independent rotation with respect to the receiver sub-assembly, together with one of multiplanar motion or monoplanar motion with respect to the receiver sub-assembly for the pivotal bone anchor assemblies.

At least one additional embodiment of the present disclosure includes non-pivoting receiver sub-assemblies in which the upper ends of the retaining structures and the lower ends of the pressure inserts are configured to form a stepped cylindrical joint when engaged together, with the retaining structures being rotatable about the vertical centerline axis of the receiver relative to the pressure inserts prior to hard locking the receiver assemblies to the shank heads.

Other additional embodiments of the present disclosure will be better understood upon review of the detailed description set forth below taken in conjunction with the accompanying drawing figures, which are briefly described as follows.

### BRIEF DESCRIPTION OF THE DRAWINGS

FIG. 1 is a partially cut-away front perspective view of a multi-component spinal fixation system showing four major types of receiver sub-assemblies and a universal bone anchor having a bi-spheric universal shank head, with each receiver sub-assembly having different functionalities and being attachable to the defined common geometry on the upper capture portion of the bone anchor, in accordance with a representative embodiment of the present disclosure.

FIG. 2 is a perspective view of the bi-spheric shank head or capture portion of the universal bone anchor of FIG. 1.

FIG. 3 is a cross-sectional side view of the universal bone anchor of FIG. 1.

FIG. 4 is a cross-sectional side view of the bi-spheric shank head or capture portion of the universal bone anchor of FIG. 1.

FIG. 5 is a perspective view of a multiplanar embodiment of a bone anchor assembly in an articulated position or configuration, in accordance with the representative embodiment of the multi-component spinal fixation system shown in FIG. 1.

FIG. 6 is an exploded perspective view of the multiplanar embodiment of the bone anchor assembly shown in FIG. 5.

FIG. 7 is a perspective view of the receiver of the multiplanar bone anchor assembly of FIGS. 5-6.

FIG. 8 is a cross-sectional side view of the receiver of FIG. 7.

FIG. 9 is a cross-sectional perspective view of the receiver of FIG. 7.

FIG. 10 is another cross-sectional perspective view of the receiver of FIG. 7.

FIG. 11 is a perspective view of the cap retainer of the multiplanar bone anchor assembly of FIGS. 5-6.

FIG. 12 is a cross-sectional perspective view of the cap retainer of FIG. 11.

Those skilled in the art will appreciate and understand that the various features and structures or components of the bone anchor assemblies shown in the drawings described above, together with their relative relationships, interconnections and functions, can be interpreted as being drawn to scale. Nevertheless, it is also understood that the representative embodiments of the present disclosure disclosed and claimed herein are not limited to the precise structures and interrelationships of the features and components shown in the drawing figures, and that the dimensions, relative positions, and interconnections between the illustrated features and components may also be expanded, reduced, re-shaped, or otherwise revised or altered as needed to more clearly illustrate the structure of the embodiments depicted therein or the functions of the various features and components, as described below. Again, it is foreseen that some parts and features are interchangeable in their arrangement between the different embodiments disclosed.

### DETAILED DESCRIPTION OF EXEMPLARY EMBODIMENTS

The following description, in conjunction with the accompanying drawings, is provided as an enabling teaching of bone anchors having a representative type of universal shank head, specifically a bi-spheric shank head or capture structure, configured for use with an array or collection of complementary pivotal and non-pivotal (i.e., multi-modal) receiver sub-assemblies in a modular spinal fixation system. The description also includes one or more methods for assembling and employing the bone anchors having bi-spheric capture structures with the multi-modal collection of receiver sub-assemblies as an advanced modular spinal fixation system for securing elongate rods to patient bone in spinal surgery. As described below, the individual bone anchor assemblies, systems, and/or methods of the present disclosure for this representative type of bi-spherical universal shank head can provide several significant advantages and benefits over other pivotal and/or non-pivotal bone anchors and spinal fixation systems known in the art due to, in one aspect, the degree of versatility and adaptability provided by the shank head universality (i.e. all of the shank heads having a common bi-spherical geometry that is connectable with each type of receiver sub-assembly having its own predetermined combination of degrees of freedom and operational functionalities) that is incorporated into the disclosed modular spinal fixation system. The recited advantages are not meant to be limiting in any way, however, as one skilled in the art will appreciate that other advantages and benefits may also be realized upon practicing the present disclosure.

Furthermore, those skilled in the relevant art will recognize that changes can be made to the disclosed embodiments for shank head universality, beyond those described, while still obtaining the beneficial results. It will also be understood and appreciated that some of the advantages and benefits of the described embodiment for the invention can be obtained by selecting some of the features (e.g., the structures or components) of the disclosed receiver sub-assemblies without utilizing other features, and that features from one sub-assembly embodiment may be interchanged or combined with features from other sub-assemblies in any appropriate combination. For example, any individual feature or collective features of method embodiments may be applied to apparatus, product or system embodiments, and vice versa. Likewise, structural elements or functional features from one embodiment may also be combined with or replaced by structural elements or functional features from one or more additional embodiments in any suitable manner. Those who work in the art will therefore recognize that many modifications and adaptations to the representative embodiments described herein are possible and may even be desirable in certain circumstances, and are to be considered part of the present disclosure for one or more inventions. Thus, it will be appreciated that the present disclosure is provided as an illustration of the principles for the representative modular spinal fixation system incorporating the bi-spherical universal shank head that are shown and discussed therein, since the scope of the invention is to be defined by the claims.

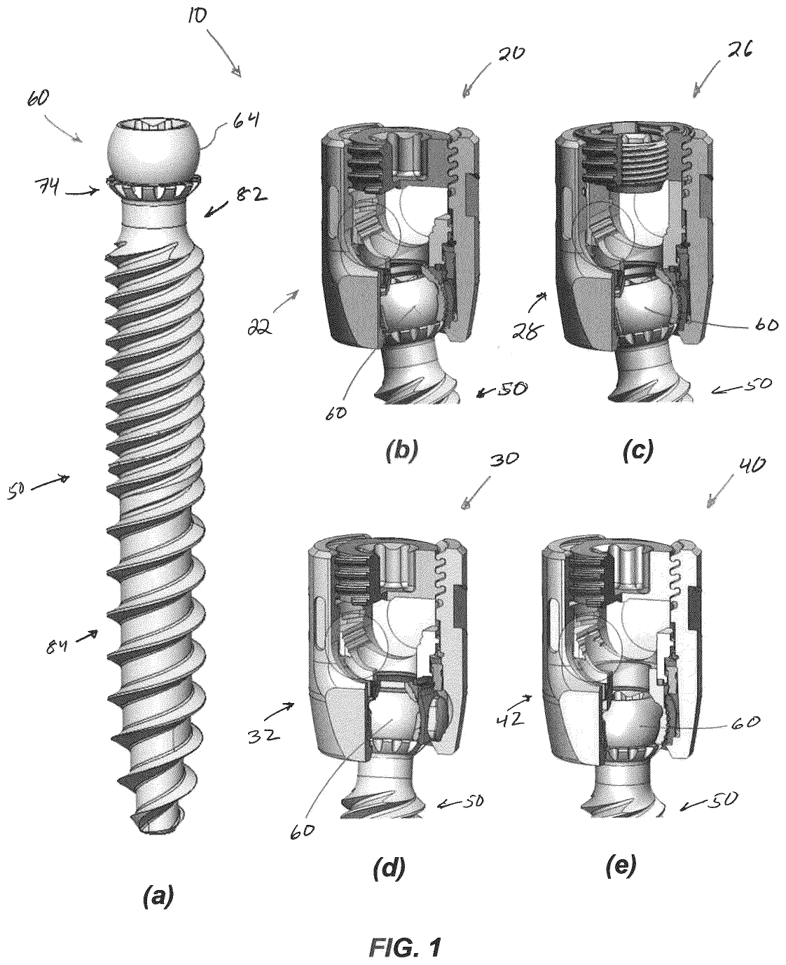

As shown in FIG. 1, the present disclosure generally relates to a modular multi-modal spinal fixation system 10 and associated methods for performing spinal fixation surgeries with the use of bone anchor assemblies having bone anchors (a.k.a. bone attachment structures such as screws, hooks, shanks, and other known anchor components) attached to longitudinal connecting members (such as rods, cords, connectors, and other known longitudinal connecting members) with bi-spherical universal shank heads 60 that can be bottom loaded into receiver sub-assemblies (i.e. housings or heads), and wherein the receiver sub-assemblies 22, 28, 32, and 42, and at least some of their associated internal components can pivot and/or rotate axially in different selected directions relative to their bone anchors. More specifically, receiver sub-assemblies that are configured to provide different mode of movement functionalities (i.e. degrees of freedom), such as multiplanar pivotal movement, monoplanar pivotal movement, and monoaxial movement (non-pivotal but axially rotatable), together with operational functionalities such as pre-lock friction fit with tool deployment of the pressure insert, pre-lock friction fit without tool deployment, provisional independent locking, open top receivers, closed top receivers, and the like, can be pre-assembled with their internal components into receiver sub-assemblies 22, 28, 32, 42 that are configured to be snapped onto or otherwise connected to the bi-spheric shank head 60 or upper end capture structure, of one or more bone anchors or shanks 50 (which may or may not be cannulated). This allows for the bone anchors to be affixed to the bony anatomy either before or after being connected with their respective pivoting or non-pivoting receiver sub-assemblies. For instance, in some cases it may be desirable to implant or attach the bone anchors or shanks 50 into or on the spine of the patient in modular fashion (i.e., independent of their larger and somewhat bulky receiver sub-assemblies), and decide later on in the surgical procedure where each of the multiplanar receiver sub-assemblies 22, 28, monoplanar receiver sub-assemblies 32, or monoaxial receiver sub-assemblies 42 should be placed and utilized on the implanted spinal construct. This type of modular, multi-modal capability can be advantageous for both midline and pedicle screw placement trajectories into the vertebral bodies and to provide for enhanced procedural solutions in certain cases, including robotic assisted surgeries.

The spinal fixation system 10 shown in FIG. 1 is directed toward eliminating or at least improving upon shortcomings of the prior art through the introduction of a bone anchor, such as a shank 50 shown in FIGS. 1(a) and 2-4, having an upper end capture structure comprising a bi-spheric or bi-spherical “universal” shank head 60 with modular, bone debris clearance, and multi-modal capabilities while being inherently free of flat side surfaces. In particular, the bi-spherical universal shank head 60 of the present disclosure is configured to be cleared of bone debris and soft tissue simultaneous with the process or motion of being “snapped” into, or otherwise connected, and captured by either a multiplanar pivotal and independently axially rotatable receiver sub-assembly 22, 28, a monoplanar pivotal and independently axially rotatable receiver sub-assembly 32, an independently axially rotatable but non-pivotal monoaxial receiver sub-assembly 42, variations on any of the above three types of receiver sub-assemblies, or any other type of receiver sub-assembly having an alternative mode of movement.

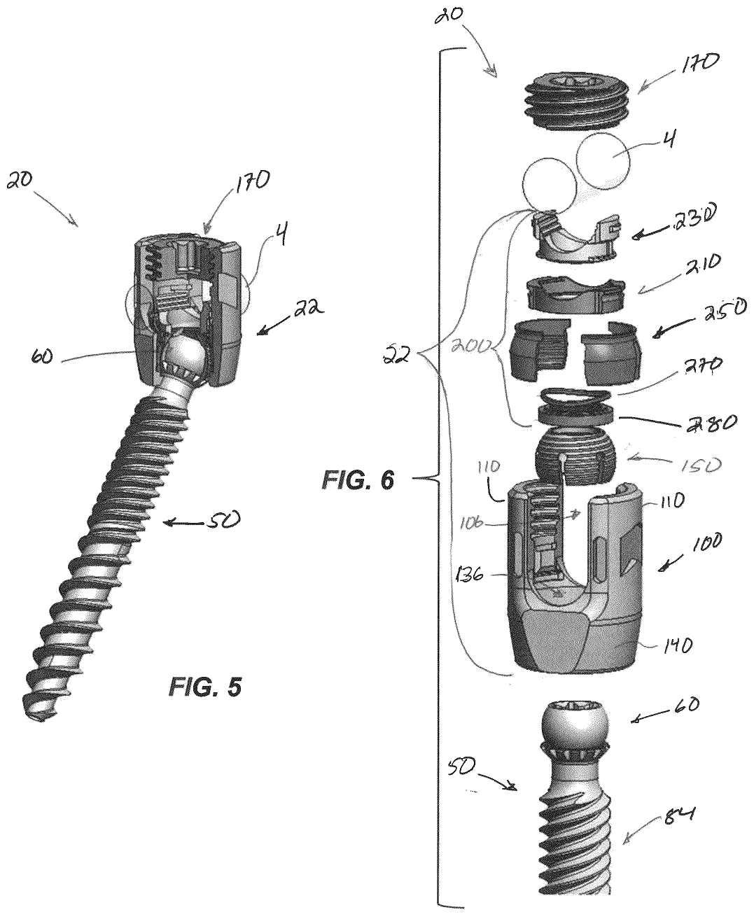

With continued reference to FIGS. 1(b) and 1(c), representative embodiments of the multiplanar pivotal and axially rotatable receiver sub-assemblies 22, 28 with bone debris clearance can be combined with the bi-spherical universal shank head of FIG. 1(a) to form multiplanar bone anchor assemblies 20, 26 further described in reference to FIGS. 5-77. With particular reference to FIGS. 1(b) and 5-63, for instance, the multiplanar bone anchor assembly 20 can include components having features or aspects configured to provide for continuous pivotal motion and rotation of the bone anchor relative to the receiver sub-assembly around a 360-degree range, and also to provide for pre-lock frictional axial rotation relative to a longitudinal axis of the bone anchor around a 360-degree range. In one aspect the multiplanar bone anchor assembly 26 can further provide for independent hard locking of the bone anchor assembly using a modified insert sub-assembly and a two-piece closure, as shown and described in reference to FIGS. 1(c) and 64-76. These embodiments of the multiplanar bone anchor assembly 20, 26 are hereinafter interchangeably referred to as a polyaxial, multi-axial, or ‘multiplanar’ bone anchor assemblies.

Similarly, the representative embodiment of the monoplanar pivotal and axially rotatable receiver sub-assembly 32, shown in FIG. 1(d), can be combined with the same bi-spherical universal bone screw to form a monoplanar bone anchor assembly 30 further described in reference to FIGS. 77-90. The monoplanar pivotal bone anchor assembly 30 can include alternative components having features or aspects configured to limit the pivotal motion of the bone anchor relative to the receiver sub-assembly (or vice versa) to a single plane (e.g., sagittal, or medial-lateral) while still providing for pre-lock frictional axial rotation around a 360-degree range, and is hereinafter interchangeably referred to as a uni-planar or ‘monoplanar’ bone anchor assembly. As shown in the drawings, the bi-spherical universal shank head can be included into this monoplanar functionality without the use of parallel flat or planar side surfaces formed into the outer surfaces of the bi-spheric shank head.

Likewise, the representative embodiment of the non-pivotal but axially rotatable receiver sub-assembly 42 shown in FIG. 1(e) can be combined with the same bi-spherical universal shank head, or upper end capture portion geometry, to form a monoaxial bone anchor assemblies 40 further described in reference to FIGS. 91-109. The monoaxial bone anchor assembly 40 can also include alternative components having features or aspects configured to prevent or inhibit pivotal motion of the bone anchor relative to the receiver sub-assembly (or vice versa) with some possible limited toggle, while still providing for pre-lock frictional axial rotation around a 360-degree range. This embodiment of the monoaxial bone anchor assembly is hereinafter interchangeably referred to as a non-pivotal or ‘monoaxial’ bone anchor assembly. Again, as shown in the drawings, the bi-spherical universal shank head 60 can be included into this non-pivotal monoaxial functionality without the use of parallel flat or planar side surfaces formed into the outer surfaces of the bi-spheric shank head.

Thus, regardless of the type, degree or amount of pivotal motion, each of the three major motion functionality embodiments of the bone anchor assembly 20, 30, 40 and their alternative embodiments or variations is configured to provide the modular multi-modal spinal fixation system 10 with the capability of rotational motion, wherein the bone anchor can at least axially rotate around its longitudinal or spin axis relative to the receiver sub-assembly 22, 28, 32, 42 (or vice versa) prior to hard locking the bone anchor assembly 20, 26, 30, 40 with a closure, and for rotation with at least some degree of a pre-lock friction fit. It will be appreciated that this feature can allow for the rotatable implantation, or screwing in, of only the anchor portion 84 of a pre-assembled bone anchor assembly to a desired depth in the bone of a patient without rotation of its respective receiver sub-assembly 22, 28, 32, 42 thereby allowing the receiver sub-assembly to be secured by separate tooling, or maintained in a desired alignment, throughout the rotatable implantation of the shank 50. This feature can also allow for the height of the receiver sub-assembly 22, 28, 32, 42 above the bone, or the length of the anchor portion 84 of the shanks 50 that is implanted in the bone, to be more precisely controlled and independently adjusted, and wherein more aggressive thread forms having larger pitches for faster insertions with fewer rotations can also be utilized, especially with robot assisted surgeries. In addition, the bi-spherical geometry of the upper end capture portion 60 of the shank 50 can further provide for a very strong and secure connection with a driving tool for navigated manual or robotic assisted screw insertions, or even direct robotic screw insertions.

Finally, it will also be appreciated each of the bone anchor assemblies 20, 26, 30, 40 of the spinal fixation system 10 shown in FIG. 1 can further provide for the re-mobilization the receiver sub-assembly 22, 28, 32, 42 relative to the bi-spheric shank head 60 after an initial hard locking of the bone anchor assembly 20, 26, 30, 40. For instance, subsequent limited unthreading or backing-off of the closure from the receiver, without removing the elongate rod or completely detaching the closure, can allow the internal components of the receiver sub-assembly 22, 28, 32, 42 to release the hard lock and re-establish a non-floppy friction fit configuration with the bi-spheric shank head 60. A slight wiggling of the receiver sub-assembly 22, 28, 32, 42, 48 can then serve to loosen or disengage the internal components so as to re-mobilize the multiplanar receiver assembly 22, 28, 32, 42, 48 relative to the bi-spheric shank head 60 and allow its position to be adjusted prior to re-locking the multiplanar bone anchor assembly 20, 26, 30, 40, 46 with a hard lock in the new position.

### Bi-Spheric Universal Bone Anchor

Referring now in more detail to the drawing figures, specifically FIGS. 1(a) and 2-4, the bone anchor or shank 50 of the spinal fixation system 10 includes the bi-spheric shank head or capture structure 60 at an upper or proximal end 51, and a body 80 extending distally from the bi-spheric shank head 60 with an attachment or anchor portion 84 at a distal end 98 configured for fixation to the bone of a patient. The body 80 of the shank 50 can be integral with the bi-spheric shank head 60 and can include a neck portion or neck 82 that extends between the bi-spheric shank head 60 and the anchor portion 84. In one aspect the neck 82 can have a cross-sectional diameter that is less than both the diameter(s) of the bi-spheric shank head 60 and the cross-sectional diameter of the anchor portion 84 immediately below the neck 82, and can be configured to pivot against an inner edge of the bottom opening of the receiver of a pivoting receiver sub-assembly 22, 28, 32 so as to provide an increased angle of articulation between the receiver and the shank 50. As shown, the anchor portion 84 can be a threaded anchor portion with one or more bone engagement threads, such as a full length dual-lead thread form 88 extending the length of the body of the shank 80 from the distal tip 96 to the neck 82, and a partial length dual-lead thread form 86 beginning at an intermediate location and extending along an upper portion of the shank body 80 to the neck 82.

The bi-spheric shank head 60 at the upper end of the shank 50 generally comprises an upper partial spherical portion 64 defining an upper spherical surface 66 that extends above and below a hemisphere plane 65 of the bi-spheric shank head 60, and a lower partial spherical portion 74 defining a lower spherical surface 76 that begins at a lower offset plane 73 that is spaced below the hemisphere plane 65 to extend downward and merge with the neck 82 of the shank body 80. A lower upward-facing shelf or annular ledge 70 extends between the upper partial spherical portion 64 and the lower partial spherical portion 74, and can be considered the portion of the lower spherical surface 76 that extends radially outward below the upper spherical surface 66. As shown in the drawings, in one aspect the annular lower ledge 70 can define an upward-facing planar ledge surface 72 that extends perpendicular to the longitudinal axis 51 of the shank along the lower offset plane 73 between the upper and lower partial spherical portions. It is foreseen, nevertheless, that the lower ledge may not extend along the lower offset plane and may instead intersect the lower offset plane and the upper edge of the lower partial spherical portion at an acute angle, thereby defining a generally upward-facing ledge surface that is frusto-conical rather than planar, whether extending upwardly and outwardly or downwardly and outwardly, from the upper partial spherical portion 64 to the lower partial spherical portion 74.

It is further foreseen that the generally upward-facing shelf surface 72 can provide an abutment face for the lower end of a driving tool, with the shelf surface being advantageously located well above the neck 82 of the shank body 80 so as to provide a shortened engagement profile for the driving tool that can reduce or substantially eliminate interferences between the driving tool and the bone of the patient when implanting the bone anchor into the bone of a patient.

Also shown in the drawings, the upper partial spherical portion 64 and upper spherical surface 66 have a minor diameter 67 that is less than the major diameter 77 defined by the lower partial spherical portion 74 and lower spherical surface 76. In one aspect the two outer spherical surfaces can be concentric, having their centers located together at the intersection between the hemisphere plane 65 and the longitudinal axis 51 of the shank. However, it is foreseen that the lower spherical surface 76 can have a center on the longitudinal axis that is offset above or below the hemisphere plane 65 defined by the upper spherical surface 66.

Moreover, the lower spherical surface 76 may not be a continuous surface, with the lower partial spherical portion 74 optionally including a plurality of open, vertically aligned flutes 78 arranged circumferentially around the bi-spheric shank head 60 and extending downwardly through and below the lower ledge 70. As described in more detail below, the flutes 78 can serve as passages and/or storage pockets for bone debris and soft tissue being pushed off the upper spherical surface 66 of the bi-spheric shank head 60 by a cap retainer during assembly of the bone anchor or shank 50 with a receiver sub-assembly. In one aspect the lower spherical surface 76 may also be considered a discontinuous lower spherical surface due to the interruptions to the spherical surface created by the flutes 78 formed into the structure of the annular lower ledge 70 and the lower partial spherical portion 74.

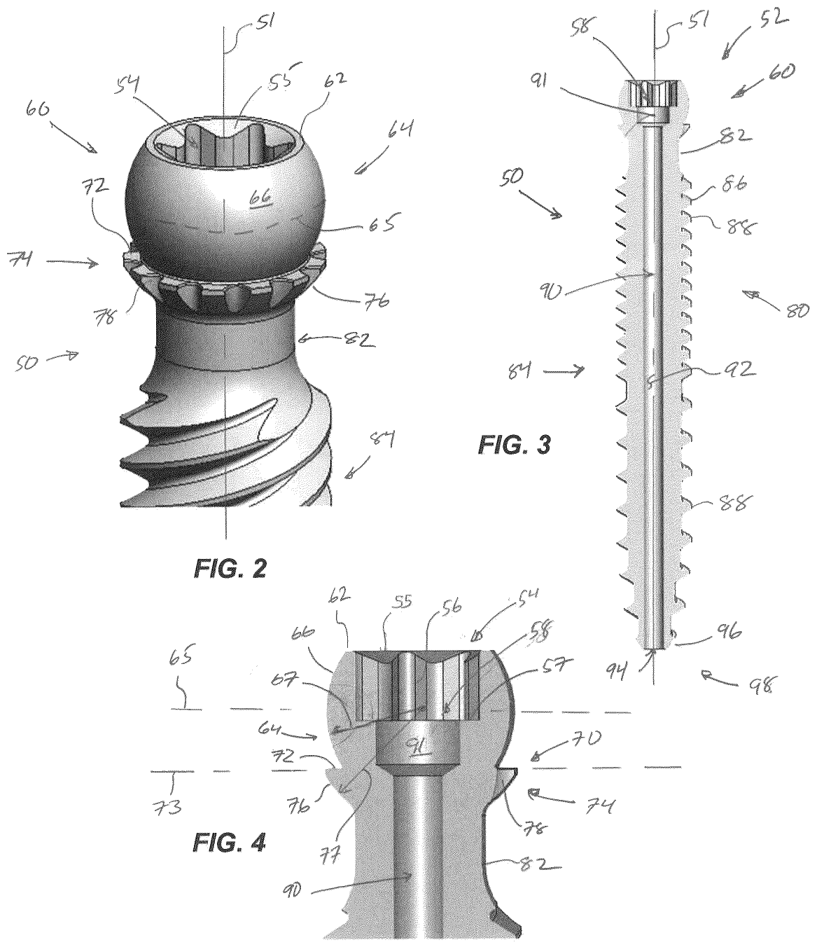

With continued reference to FIGS. 2-4 the bi-spheric shank head can have an annular planar top surface 62 that surrounds an internal drive feature or drive socket 54. For example, the internal drive feature 54 of the shank 50 illustrated in the figures is an aperture formed in an inwardly-tapered upper surface 55 that is surrounded by the annular planar top surface 62. In one aspect the internal drive feature 54 may be a multi-lobular or star-shaped aperture, such as those sold under the trademark TORX, or the like, having vertically-aligned sidewalls or internal faces 56 designed to receive a multi-lobular or star-shaped tool for rotating and driving the shank body 80 into the vertebra. It is foreseen that such an internal drive feature 54 may take a variety of tool-engaging forms and may include one or more apertures of various shapes, such as a pair of spaced apart apertures or a hex shape designed to receive a hex tool (not shown) of an Allen wrench type. A seat or base surface 57 of the internal drive feature 54 can be disposed perpendicular to the shank longitudinal axis 51, with the internal drive feature 54 otherwise being coaxial with the shank longitudinal axis. In operation, a driving tool is received in the internal drive feature 54, being seated at the base surface 57 and engaging the internal faces 56 of the internal drive feature 54 for both driving and rotating the anchor portion 84 of the shank body 80 into the vertebra, either before or after the shank 50 is attached or coupled to a multiplanar receiver sub-assembly. If attached, the threaded anchor portion 84 of the shank body 80 can be driven into the vertebra with the driving tool extending into and through the receiver.

Also shown in the drawings, in some embodiments the shank 50 can be cannulated with an axial bore 90 extending through the length thereof and centered about the longitudinal axis 51 of the shank 50. The axial bore 90 can be defined by an inner cylindrical wall 92 of the shank having a circular opening 94 at the distal tip 96 and an upper opening 58 communicating with the internal drive socket 54 at the seat or base surface 57 of the drive socket 54 (FIGS. 3 and 4). The axial bore 90 can further include an upper expanded portion 91 just below the upper opening 58 with the drive socket 54 that can, in one aspect, be configured to accommodate tooling, such as the distal stub portion of a drive or centering tool (not shown). The axial bore 90 is generally coaxial with the shank body 80 and the bi-spheric shank head 60, and can provide a passage through the shank interior for a length of wire (not shown) to provide a guide for insertion of the shank body 80 into the vertebra. The axial bore 90 of the cannulated shank can also provide for a pin to extend therethrough and beyond the shank tip, the pin being associated with a tool to facilitate insertion of the anchor portion 84 of the shank body 80 into the vertebra.

### Multiplanar Bone Anchor Assembly

FIG. 5 is partially-sectioned perspective view of one representative embodiment of the complete multiplanar bone anchor assembly 20 illustrated in FIG. 1(b), with the multiplanar receiver sub-assembly 22 and an elongate rod 4 being connected to the bi-spheric shank head 60 of the bone anchor or shank 50 with a closure 170, and with the shank 50 being pivoted and locked or ‘hard’ locked at an angle with respect to the receiver of the receiver sub-assembly 22. FIG. 6 is an exploded perspective view of the same multiplanar bone anchor assembly 20 and rod 4. As described above, the bone anchor or shank 50 of the multiplanar bone anchor assembly 20 includes the bi-spheric shank head or capture structure 60 at an upper or proximal end of the shank, and an anchor portion 84 opposite the bi-spheric shank head that is configured for securement within or attachment to the bone of a patient (not shown).

With continued reference to FIG. 6, the multiplanar receiver sub-assembly 22 generally includes a housing or receiver 100 having a base portion 140 defining an internal cavity 136 or lower portion of a central bore that is configured to accommodate a pivoting or articulating multiplanar cap retainer 150 that is couplable or fixable to the bi-spheric shank head 60, and a pair of upright arms 110 extending upwardly from the base portion 140 to define a rod channel 106 that is configured to receive the elongate rod. As discussed in more detail below, the central bore communicates with a bottom surface of the base 140 of the receiver 100 through a bottom opening, and extends upwards through the rod channel 106 to the top of the receiver (or tops of the upright arms when the rod channel is an open channel, as shown in the drawings).

The receiver 100 can be initially pivotally secured to the bi-spheric shank head 60 with a number of separate internal components that have been pre-assembled into the central bore and the rod channel to form the multiplanar receiver sub-assembly 22. These internal components generally include the multiplanar cap retainer 150 and a multiplanar insert sub-assembly 200. As described above, the multiplanar cap retainer 150 can be positioned in the internal cavity or lower portion of the central bore of the receiver, and attaches to the bi-spheric shank head 60 to pivotally couple the shank 50 to the receiver sub-assembly 22. The multiplanar insert sub-assembly 200 can, in turn, be comprised a plurality of additional components, including but not limited to a central support collar 210 that is coupled around the lower end of a load saddle 230 and the upper end of a two-piece clamp positioner 250, with an additional wave washer 270 and a crown element 280 being uploaded through a lower opening of the clamp positioner 250 and into the center aperture of the support collar 210 to complete the multiplanar insert sub-assembly 200. The multiplanar insert sub-assembly 200 can also be a resiliently axially biasing sub-assembly, as described in more detail below. After an elongate rod 4 has been positioned within the lower portion of the rod channel, a single-piece closure 170 can be threadably or otherwise secured into an upper portion of the rod channel to apply pressure to an upper surface of the rod, such as by direct contact, thereby locking both the elongate rod 4 and the multiplanar bone anchor assembly 20 into a hard locked position that may or may not be final.

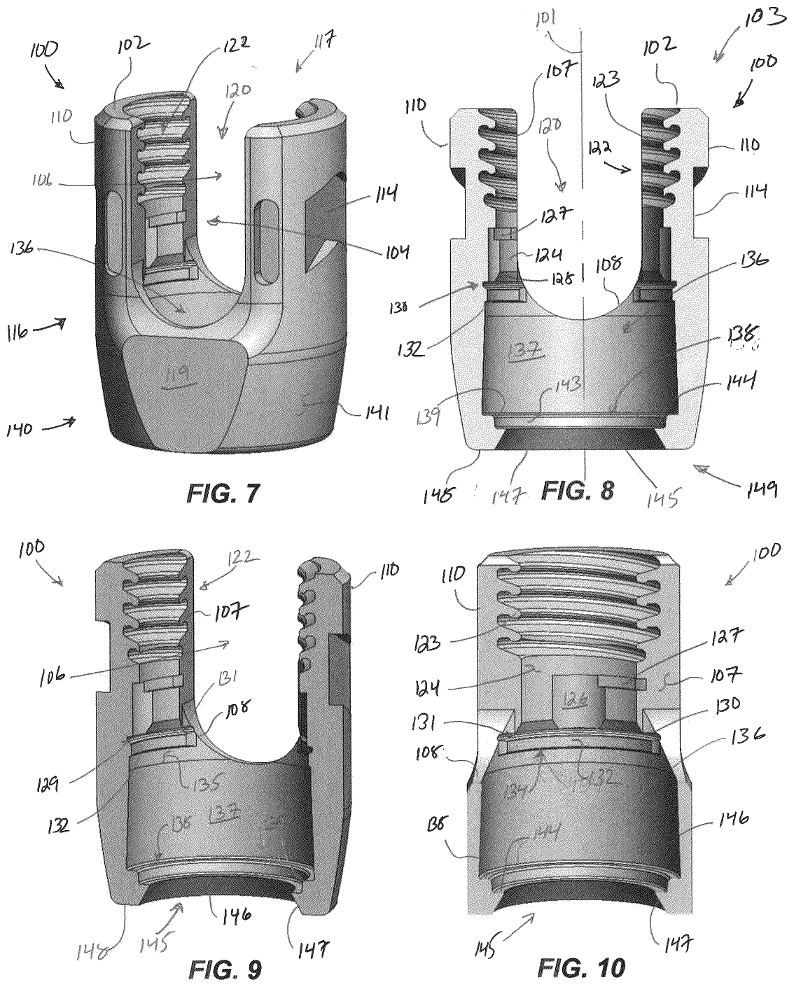

Illustrated in FIGS. 7-10 is the multiplanar embodiment of the receiver 100 having a base 140 defining an interior cavity 136 or lower portion of a central bore 120 that communicates with a bottom surface 148 of the receiver 100 through a bottom opening 145, and a pair of upright arms 110 extending upwardly from the base 140 to define a rod channel 106 that receives the elongate rod. In one aspect the rod channel 106 can be an open channel, such as that shown in the drawings, but other configurations for the upper portion of the receiver, including a closed channel defined by a solid or integral ring structure at the upper end of the receiver, are also contemplated. The interior cavity 136 or lower portion 136 of the central bore 120 can be defined by sidewalls 137 that taper downwardly and outwardly to a stepped seating structure that includes an annular locking recess 142. The tapered sidewalls 137 can provide clearance for expansion and movement of the internal components of the receiver sub-assembly 22. In one aspect the central bore 120 can further include opposed shipping state grooves 130 and opposed horizontal ridges 132 formed into the inner surfaces of the central bore 120 above the internal cavity 136.

As illustrated, the multiplanar receiver 100 can have a generally U-shaped appearance with a partially-discontinuous and substantially-cylindrical inner profile, and a partially-cylindrical and partially-faceted outer profile, although other profiles are also contemplated. For example, it is foreseen that this type of receiver can also be configured with planar lateral side surfaces. As described above, the receiver 100 generally comprises the base portion 140 defining the internal cavity 136 or lower portion of a generally cylindrical central bore 120 that is centered around the receiver's vertical centerline axis 101, and the pair of upright arms 110 extending upwardly from the base 140 to form the upper portion of the receiver and to define the upwardly-open channel 106 that is configured for receiving the elongate rod. Each of the upright arms 110 has an interior face 104 that includes a discontinuous upper portion of the central bore 120, which may be bounded on either side by vertically-aligned opposed planar surfaces 107 that curve downwardly into lower saddle surfaces 108, which can be U-shaped. In one aspect the opposed planar surfaces 107 and the curved saddle surfaces 108 can together define the front and back ends of the upwardly open channel 106 that opens laterally onto the front face 116 and the back face 117 of the receiver 100, respectively. From the top surfaces 102 of the upright arms 110 at the proximal end 103 of the receiver, the central bore 120 can extend downwardly through both the open channel 106 and the internal cavity 136 to communicate with a bottom surface 148 of the receiver through a bottom opening 145 at the distal end 147 of the receiver.

The upper or channel portion of the central bore 120 further includes a discontinuous guide and advancement structure 122 formed into the interior faces 104 of the upright arms 110, which guide and advancement structure 122 is configured to engage with a complementary structure formed into the outer side surfaces of the closure 170 (see FIGS. 5-6), as described more fully below. The guide and advancement structure 122 in the illustrated embodiment is a discontinuous, helically-wound interlocking flange form 123. It will be understood, however, that the guide and advancement structure 122 could alternatively comprise a square-shaped thread, a buttress thread, a modified buttress thread, a reverse angle thread, or other thread-like or non-thread-like closure mating structure for operably guiding the closure downward between the upright arms 110 under rotation until the closure directly engages and presses against the elongate rod positioned within the channel 106. Additionally, the various structures and surfaces forming a helically wound guide and advancement structure 122 can also be configured to resist, to inhibit, to limit, or to preferentially allow and control some limited amount of splay of the upright arms 110 of the receiver 100 while advancing the closure downward under rotation and when torquing the closure against the elongate rod to generate a downwardly-directed thrust that locks the completely assembled multiplanar bone anchor assembly into position (see FIG. 5).

Moving downward along the interior faces 104 of the upright arms 110, the portion of the central bore 120 located between the vertically-aligned opposed planar surfaces 107 and below the guide and advancement structure 122 can include an upper discontinuous cylindrical surface 124 having an inner diameter that, in one aspect, can be substantially equal to the crest diameter of the helically-wound interlocking flange form 123. Alternatively, it is foreseen that the inner diameter of the upper discontinuous cylindrical surface 124 may be greater than or less than the crest diameter of the flange form 123, and that a run-out groove or grooves may also be formed into the interior faces 104 of the upright arms between the guide and advancement structure 122 and the upper discontinuous cylindrical surface 124

With continued reference to FIGS. 7-10, the upper discontinuous cylindrical surface 124 can further include opposed vertically-elongate side pockets 126, with each side pocket 126 being defined by a vertically-aligned, inwardly-facing sidewall surface that can be bounded above and below by upper and lower planar surfaces, respectively. As described in more detail below, the sidewall surface can be sized and shaped to generally match the profile of opposite indexing structures or nubs extending from the load saddle 230 of the insert sub-assembly 200. Furthermore, as shown in the drawing figures, both the side pockets 126 and the indexing nubs can have arc-shaped profiles.

Each side pocket 126 can further include one or more horizontal access recesses 127 extending from an upper portion of the side pocket 126 to an opposed planar surfaces 107 of the upright arm 110, so as to provide access to the side pockets 126 for the indexing nubs. In one aspect the depth of the horizontal access recesses 127 relative to the upper discontinuous cylindrical surface 124 can vary, and in particular can become slightly reduced or shallower, ramped, or increasingly-inwardly-sloped as moving from the opposed vertical planar end surfaces 107 toward the side pockets 126. This slight reduction in depth can create a resistance to the movement of the indexing nubs through the access recesses, and correspondingly a resistance to the rotation of the insert sub-assembly 200 about the centerline axis 101 of the receiver 100, and which resistance can be released as soon as the indexing nubs pass completely through the horizontal access recesses 127 and into the vertical side pockets 126. It will be appreciated that the reduced depth of the horizontal access recess 127 as it merges with the side pocket 126 can also serve to inhibit the indexing nubs from accidentally or unintentionally re-entering the horizontal access recesses 127 from within the side pocket 126 after the load saddle 230 of the insert sub-assembly 200 has been rotated into its aligned position with the channel 106 of the receiver 100.

The lower end of the upper discontinuous cylindrical surface 124 ends in a short tapered surface 128 that extends downwardly-and-outwardly to opposed shipping state grooves 130 located toward the lower end of the channel 106. In one aspect the shipping state grooves 130 can extend across the width of the interior faces 104 of each upright arm 110 to the lower saddle surfaces 108 at both the front face 116 and the back face 117 of the receiver 100. As described below, this configuration of the shipping state grooves 130 can allow for opposite upper outer rim structures of the support collar 210 to be rotated into position within the shipping state grooves 130 from either direction. It is foreseen that other configurations for the opposed shipping state grooves are possible, including shipping state grooves with a center portion that only extends in one rotational direction (i.e., clockwise or counter-clockwise) to one of the lower saddle surfaces 108, such that the opposite upper outer rims of the support collar 210 can only be rotated into position from that direction.

The upper surfaces 129 of the opposed shipping state grooves 130 can be downward-facing arcuate planar surfaces configured to engage with planar upper surfaces of the opposite outer flange structures, so as to prevent the support collar 210 from moving upward within the central bore 120 after the opposite upper outer rims have been rotated into position within the shipping state grooves 130. In contrast, the lower surfaces of the shipping state grooves 130 can comprise ramped surfaces 131 that extend downwardly and inwardly toward the partially-cylindrical top surfaces 133 of opposed horizontal ridges 132 located just below the shipping state grooves 130. The underside surfaces 134 of the opposed horizontal ridges 132 can also be downward-facing arcuate planar surfaces configured to engage with the same planar upper surfaces of the opposite upper outer rims of the support collar 210 after the support collar has been pushed downwardly across the axial width of the top surfaces 133 of the opposed horizontal ridges 132, as described in more below.

Moving downward through the central bore 120, a lower discontinuous cylindrical surface 135 can be located below the opposed horizontal ridges 132, and can extend a short distance downward until reaching the upper extent of a frustoconical or tapered sidewall surface 137 that defines the central and upper portions of the internal cavity 136 of the receiver 100. The sidewall surface 137 can taper downwardly and slightly outwardly until reaching an annular, upward-facing upper step surface 138 that defines the lower end of the expansion portion of the internal cavity 136. Adjacent to and below the upper step surface 138 is an annular locking recess 142 defined by a lower sidewall surface 143 and an annular, upward-facing lower step surface 144. In one aspect a beveled or chamfered surface 139 can extend between the upper step surface 138 and the lower sidewall surface 143 to help guide lower edges of the two-piece clamp positioner 250 into the annular locking recess 142, as will be described in more detail below.

Below the lower step surface 144 of the annular locking recess 142 is a frustoconical surface 147 that extends downwardly and outwardly to the substantially planar bottom surface 148 at the distal end 149 of the receiver 100. As illustrated, the bottom opening 145 of the receiver can be defined by the relatively-sharp circular edge 146 between the lower step surface 144 and the lower-most frustoconical surface 147, with the later providing a tapered approach to the bottom opening 145. Nevertheless, it is foreseen that other configurations for the defining the bottom opening of the receiver, such as a narrow cylindrical surface, are also possible and considered to fall within the scope of the present disclosure.

As described above, the multiplanar receiver 100 can have a partially cylindrical and partially faceted outer profile. In the illustrated embodiment, for example, the partially cylindrical portions can include curvate side outer surfaces 112 of the upright arms 110 opposite the interior faces 104 that extend downward from the top surfaces 102 of the upright arms toward a lower outer tapered surface 141 of the base 140 that can angle inwardly to the bottom surface 148 of the receiver 100. The receiver 100 can further include upper curvate-extending instrument engaging grooves 114 below the top surfaces 102 of the upright arms 110 that extend horizontally across the curvate side outer surfaces 112, and in one aspect (not shown) can extend to the front face 116 and the back face 117 of the receiver 100.

Likewise shown in the drawings, the faceted or planar portions of the receiver 100 may comprise front and back outer planar faces 119 on the receiver base 140 below the open channel 106, as well as narrow flats, recesses, or tool engagement features on the front and back faces 116, 117 of the upright arms. The faceted or planar portions of the multiplanar receiver 100 can further include side outer planar faces (not shown) and/or tool receiving and engaging recesses (also not shown) formed into the curvate side outer surfaces 112 below the upper instrument engaging grooves 114, and which can be parallel with each other and oriented perpendicular to the front and back outer planar faces 119. In one aspect the upper instrument engaging grooves 114, the front and back outer planar faces 119, the narrow flats or tool engagement features, and any other planar tool-engagement surface or recess can serve together as outer tool engagement surfaces that allow for tooling to more securely engage and hold the receiver 100 during an initial pre-assembly with the internal components to form the multiplanar receiver sub-assembly 22, during coupling of the receiver sub-assembly to the bone anchor (either after or before the implantation of the anchor portion 84 of the bone anchor into a vertebra), and also during further assembly of the multiplanar receiver sub-assembly 20 with the elongate rod and the closure so as to aid in torquing and counter-torquing to lock the assembly.

Furthermore, it will be appreciated that the receiver 100 can also include additional features and aspects not shown in the drawings, including but not limited to inwardly-threaded breakoff extensions extending upwardly from the tops of the upright arms for interfacing with tooling and for guiding the elongate rod and the outwardly-threaded closure into the receiver channel. It is also foreseen that other shapes and configurations for the interior and exterior surfaces of the receiver 100, different from those shown in the drawings while providing for similar interaction and functionality of the various components of the pivotal bone anchor assembly, are also possible and considered to fall within the scope of the present disclosure, including but not limited to receivers having bottom openings with cut-out sections or slanted bottom surfaces that form oblique or expanded bottom openings, and the like, that provide for increased pivotal motion for the shank in at least one direction.

It is also foreseen that in alternative embodiments of the present disclosure the receiver can be configured with a closed rod-receiving channel, in which case the top surfaces and upper portions of the upright arms can be connected together to form a solid ring surrounding the central bore, and in which case one or more of the internal components of the sub-assembly can be uploaded into the central bore of the receiver through its bottom opening. In one such a bottom-loaded embodiment, for instance, it will be appreciated that the seating surface of the internal cavity can be replaced with an internal recess located adjacent the bottom opening that is configured to receive a separate open retaining ring having a slit or slot to provide for the contraction and expansion thereof, with the open retainer ring having an upper inner edge or partially spherical inner surface that is configured, in turn, to engage and support the lower spherical surface of the bi-spheric shank head and the cap retainer coupled to the bi-spheric shank head. Other configurations for the closed-top receiver sub-assembly and/or for the bottom-loaded components are also possible and considered to fall within the scope of the present disclosure.

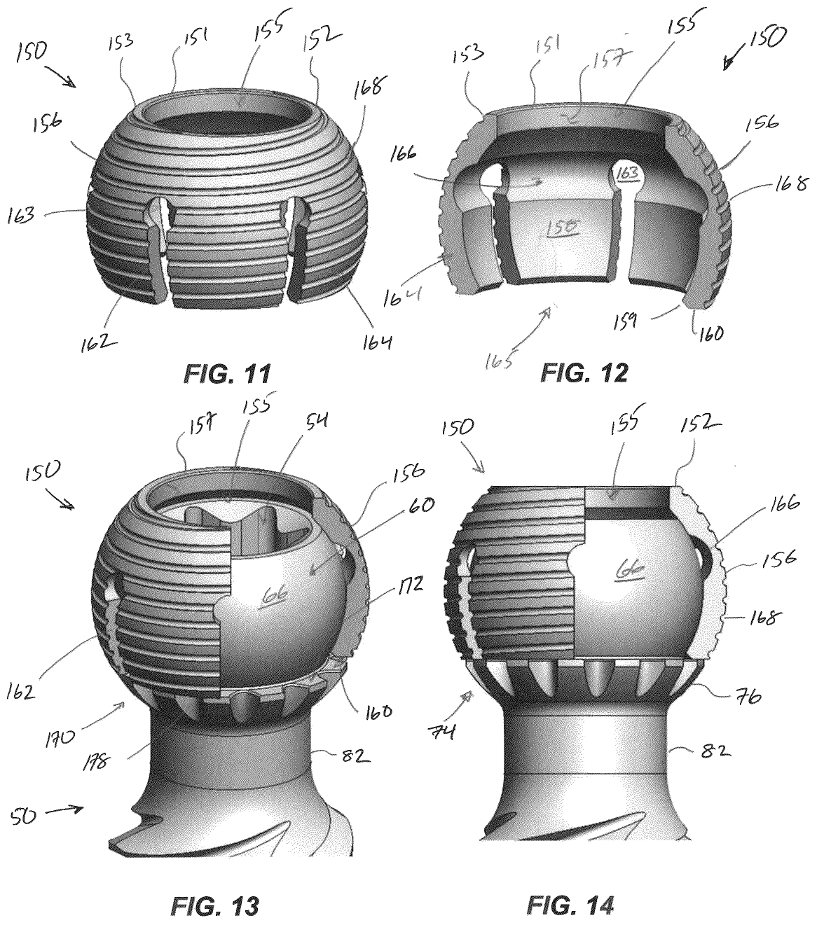

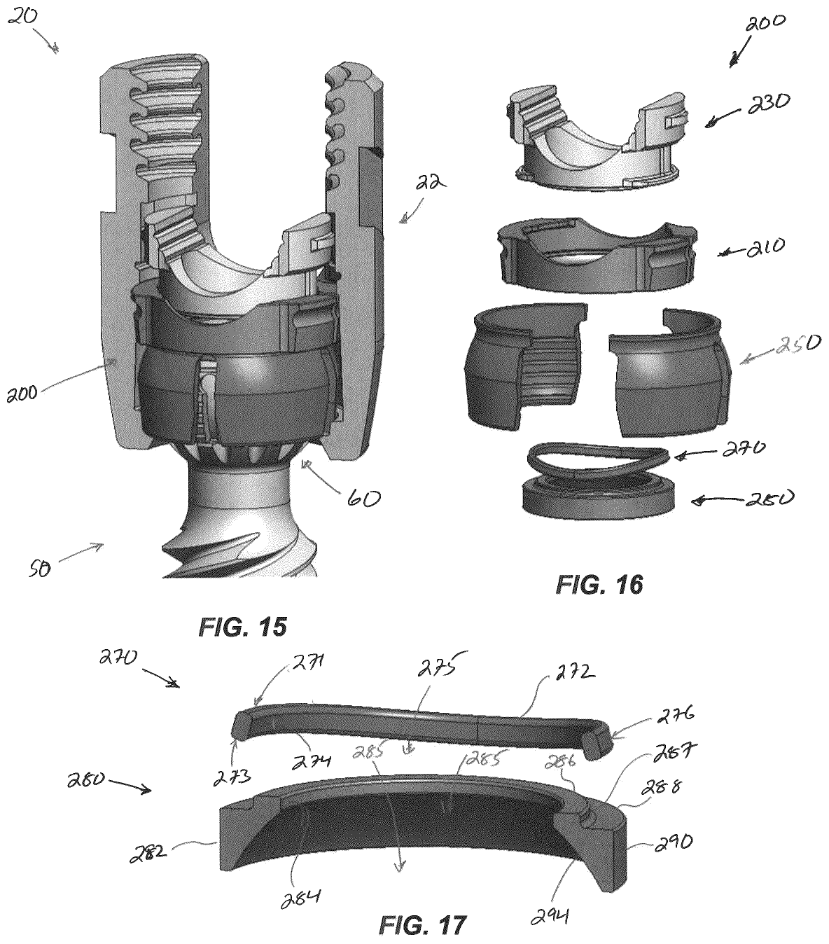

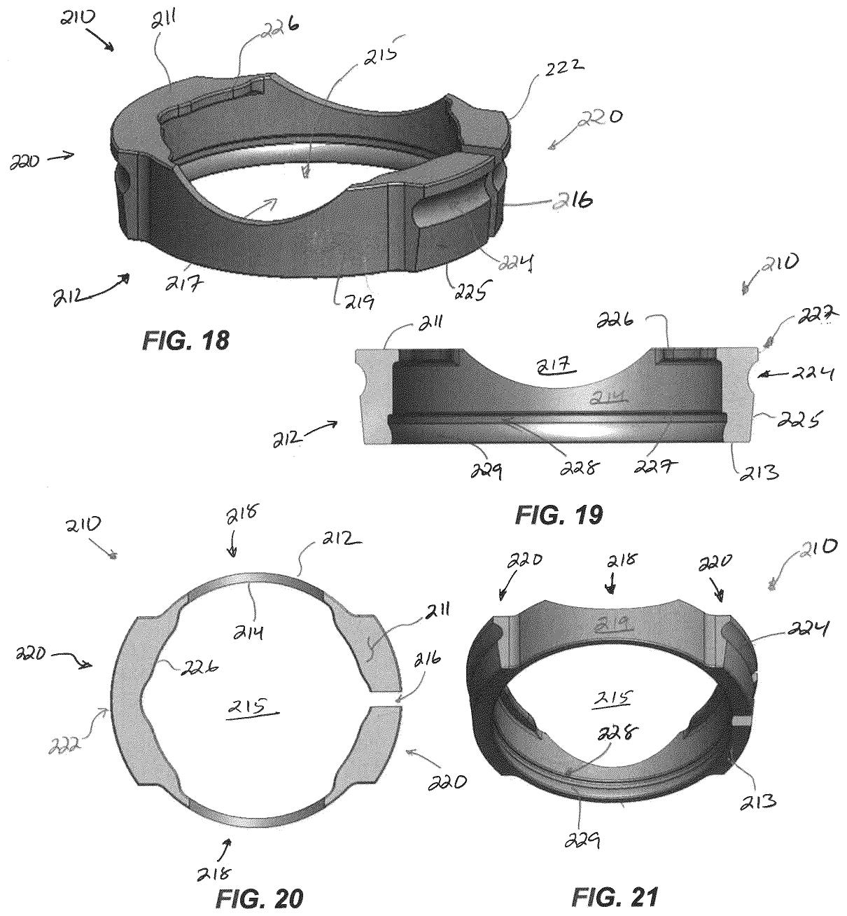

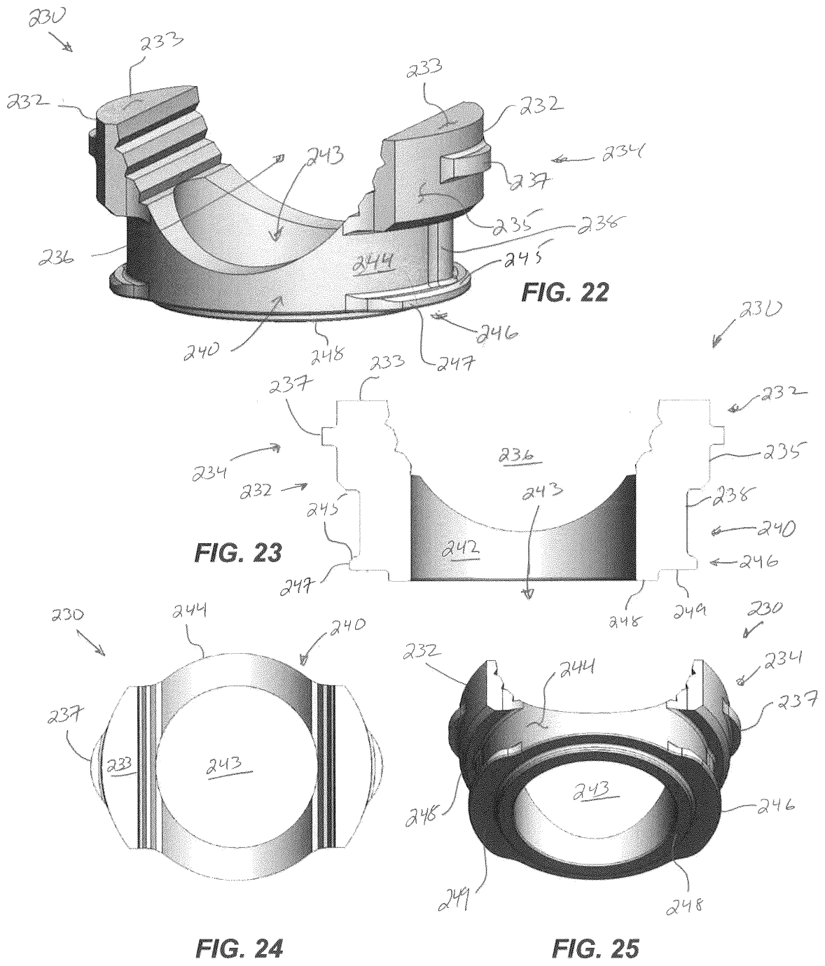

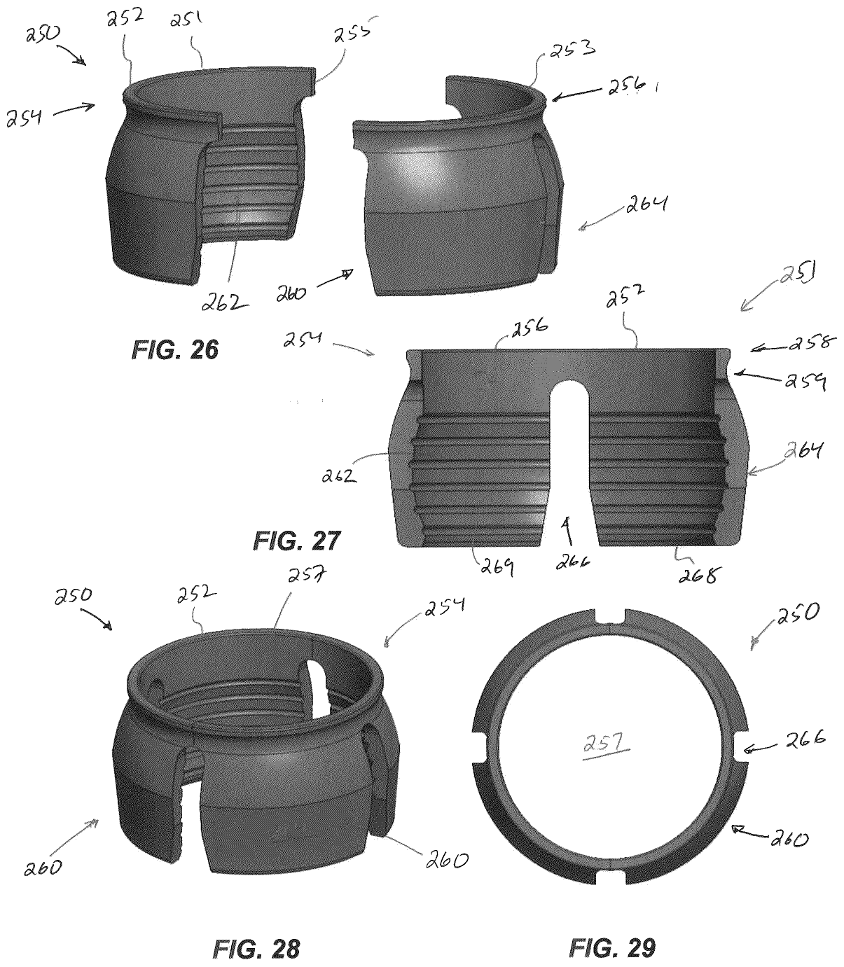

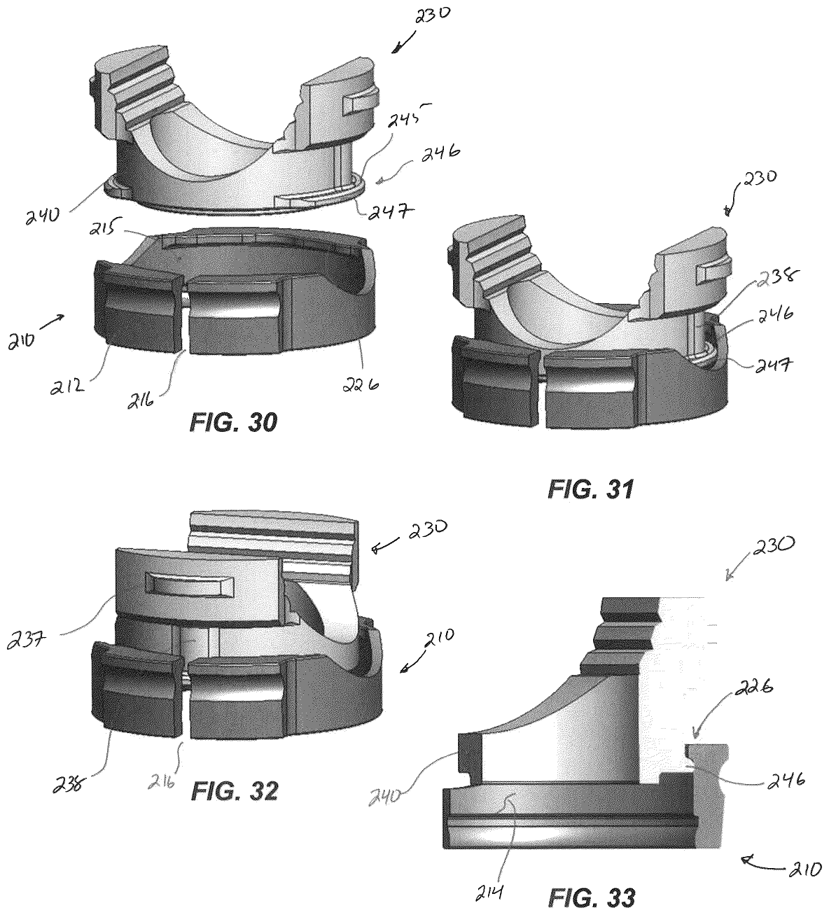

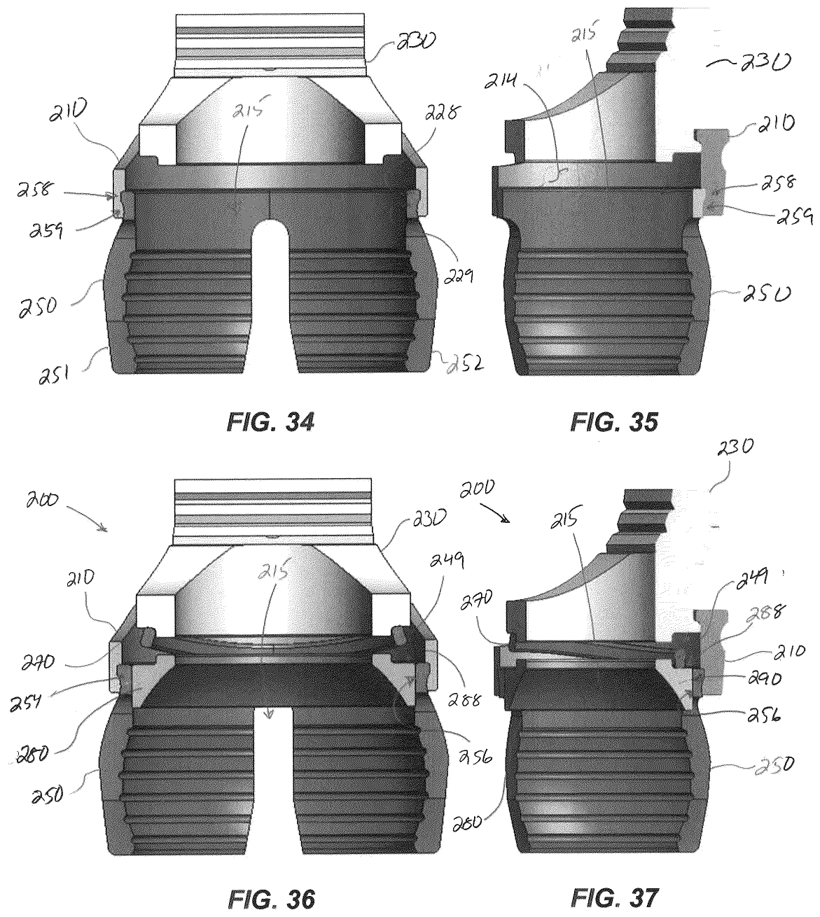

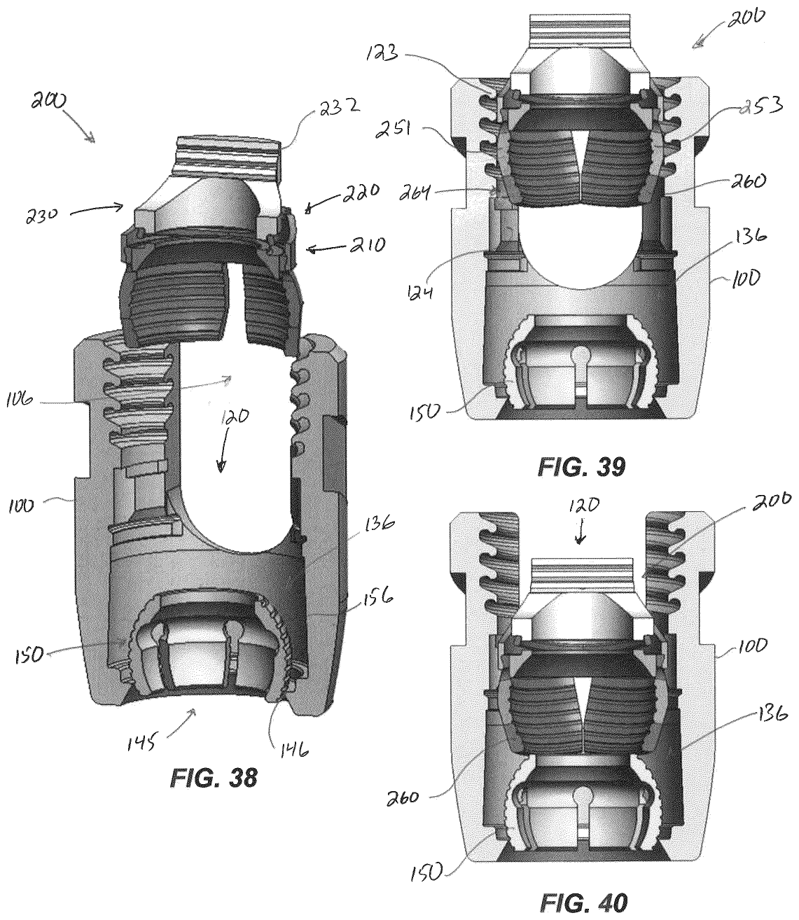

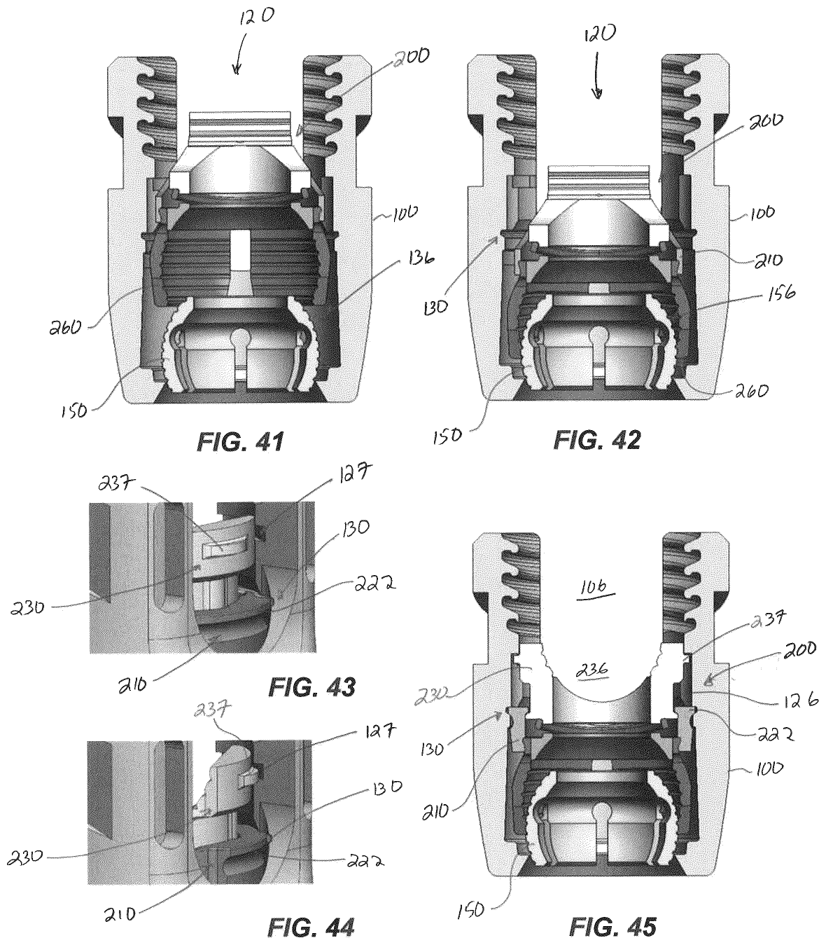

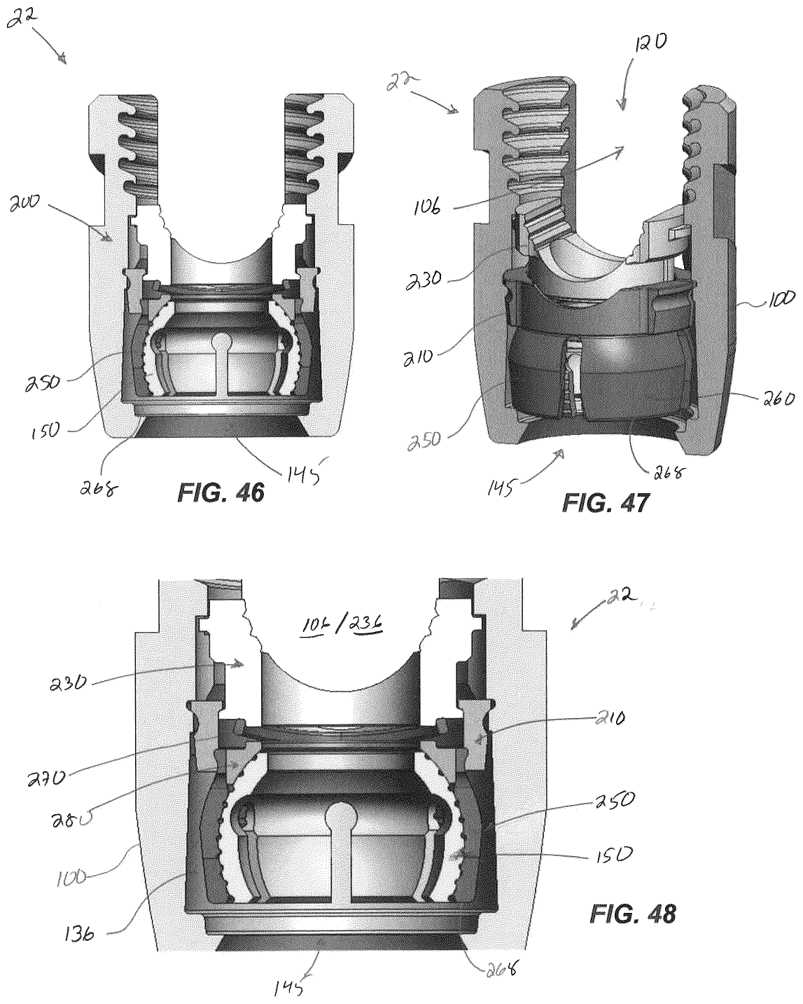

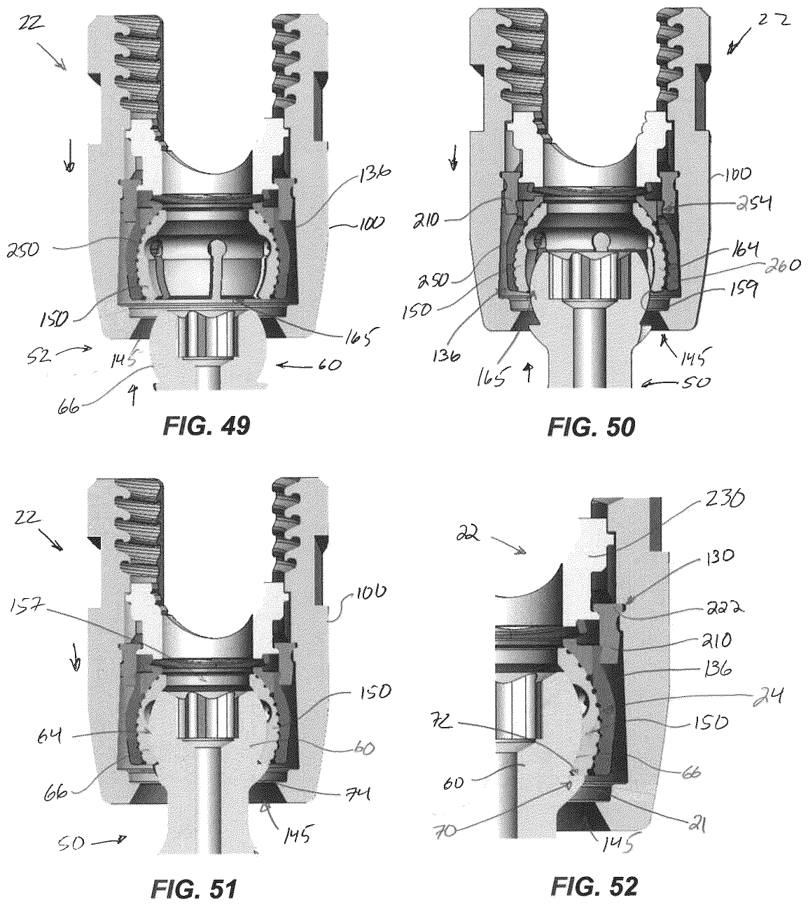

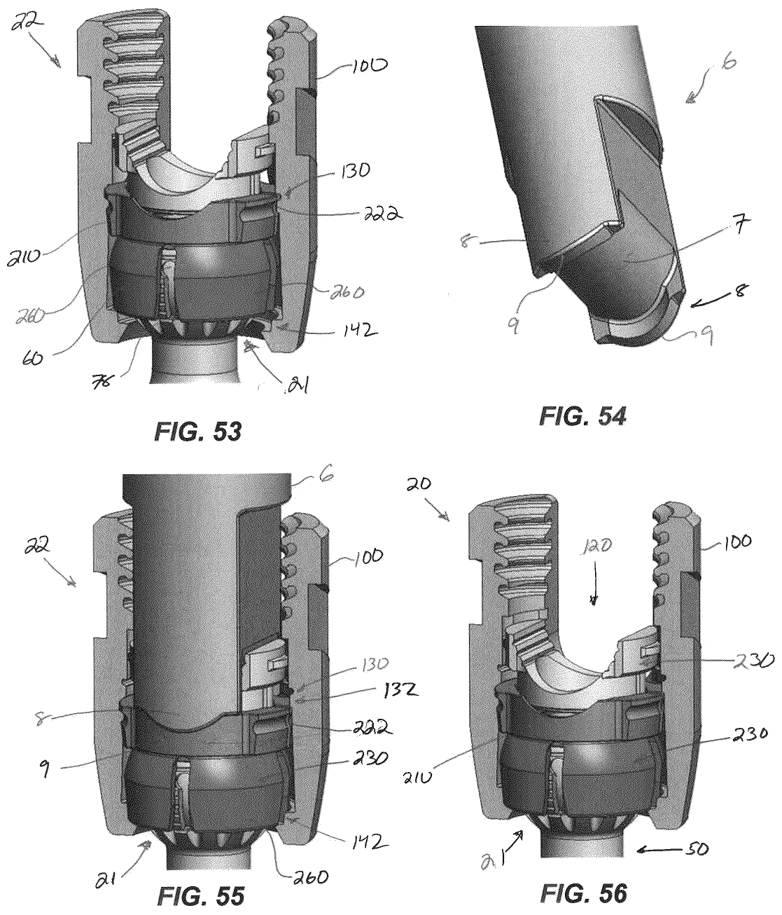

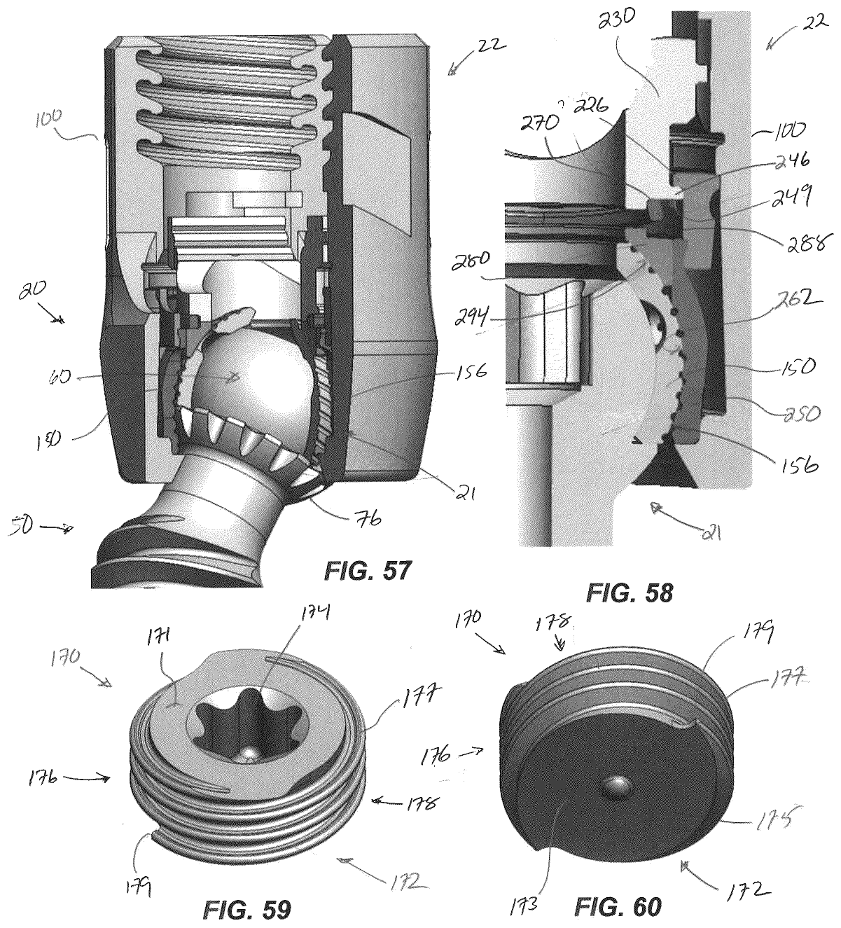

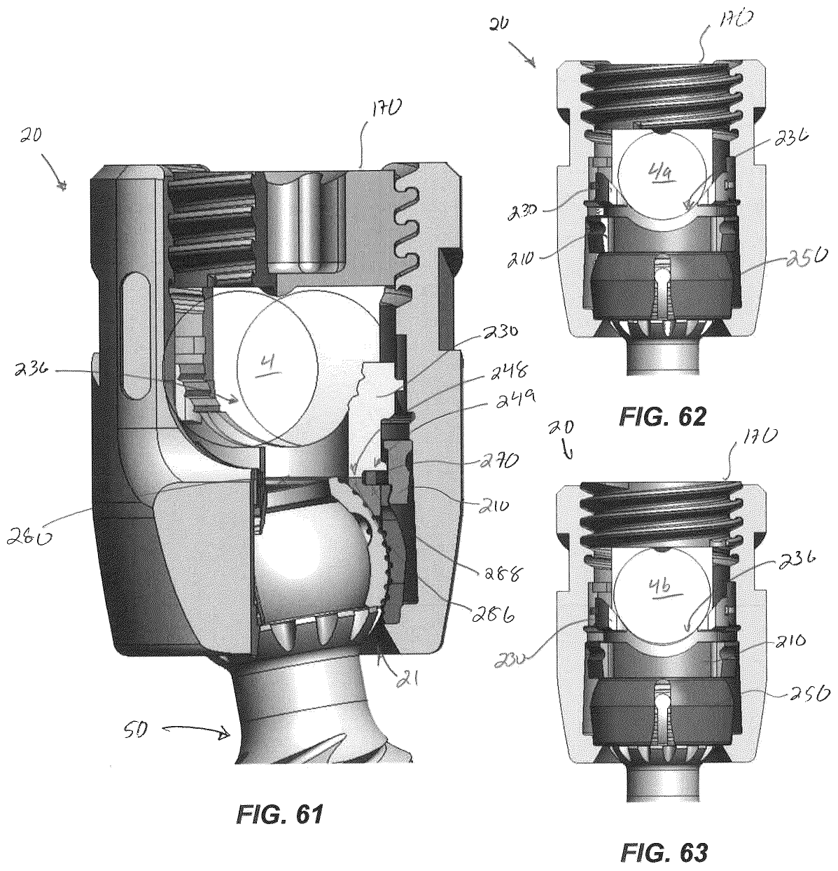

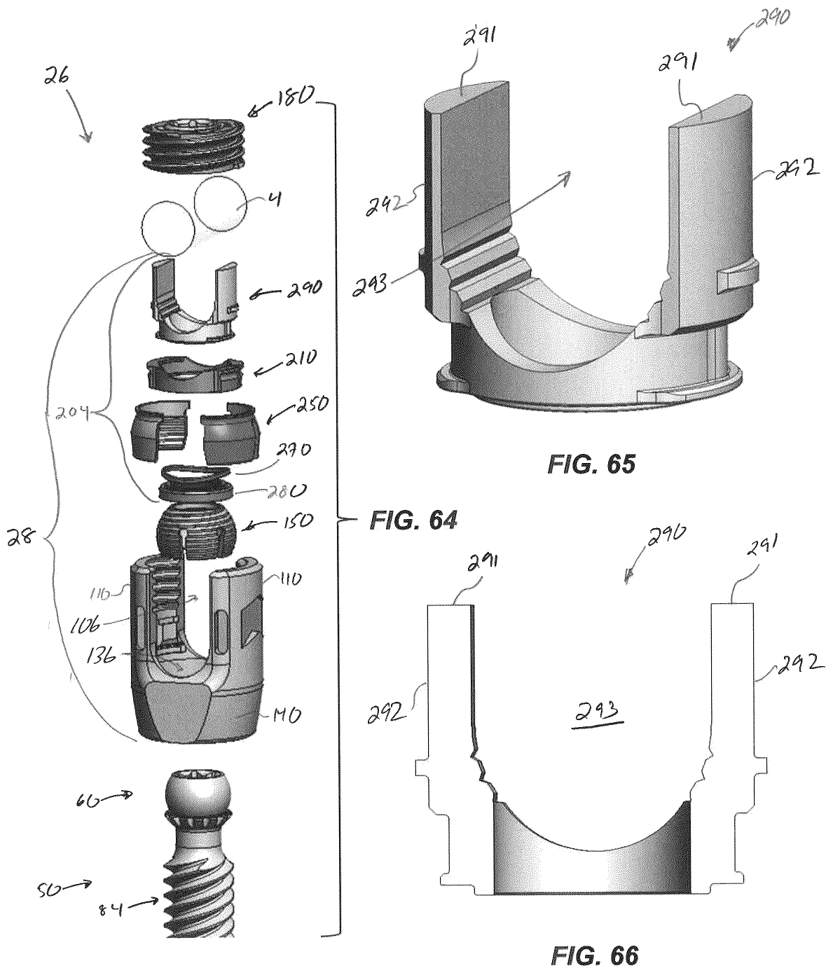

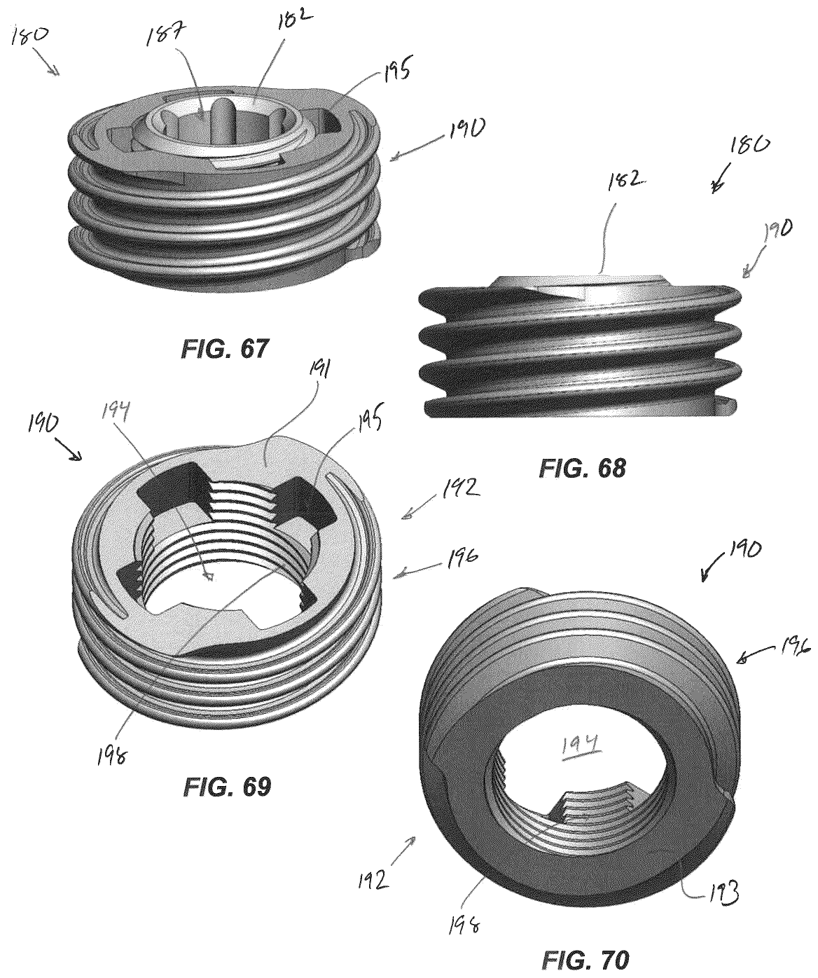

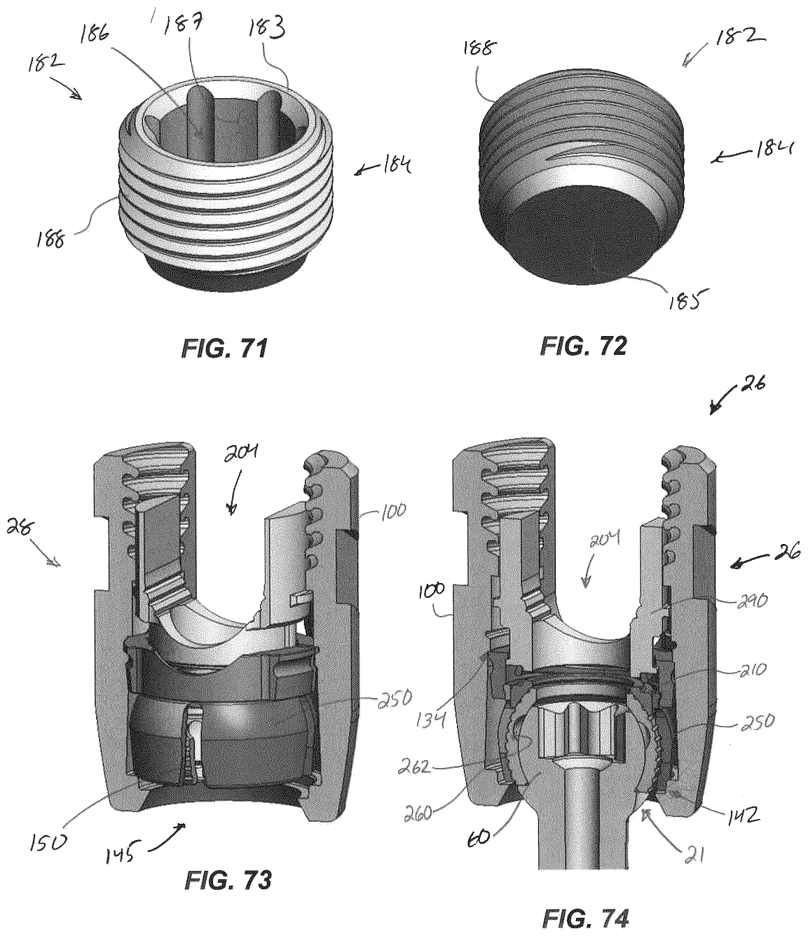

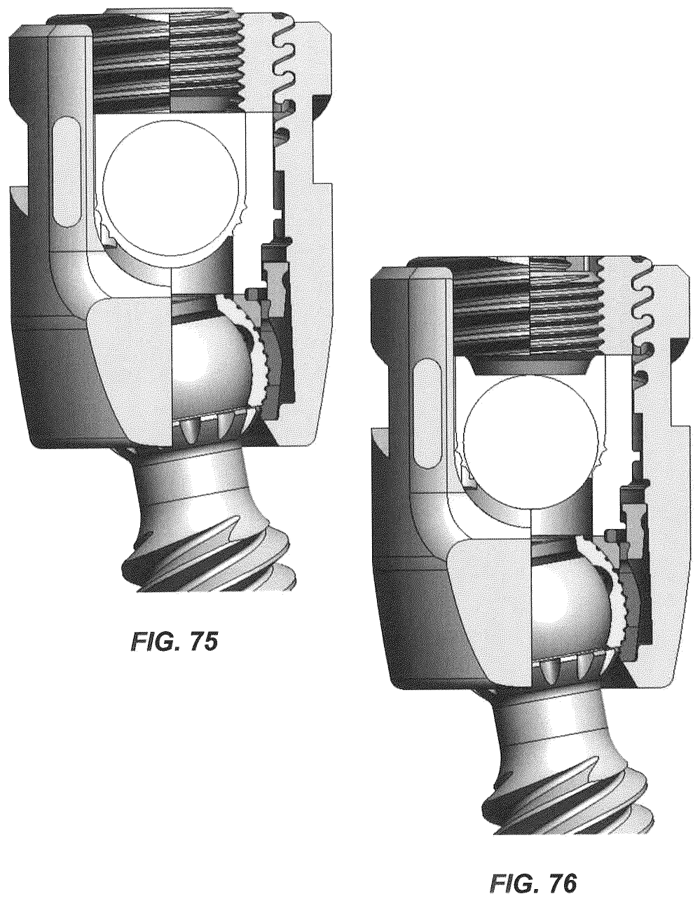

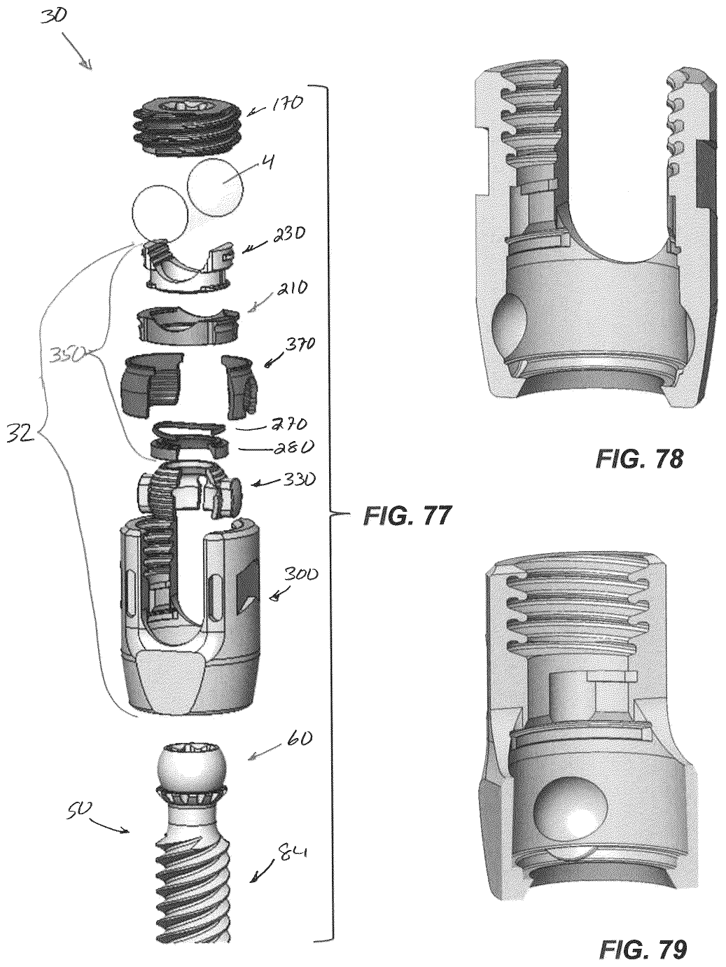

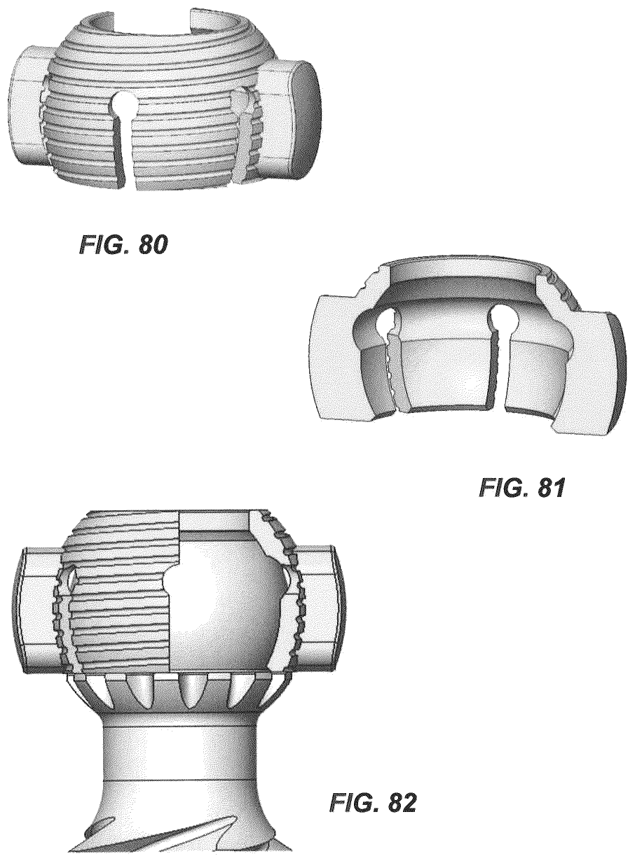

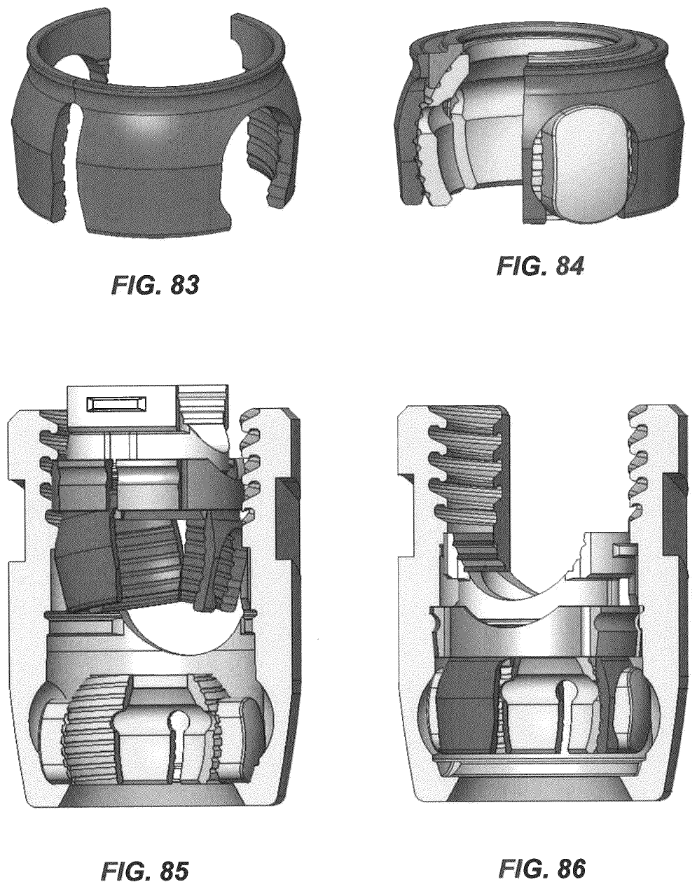

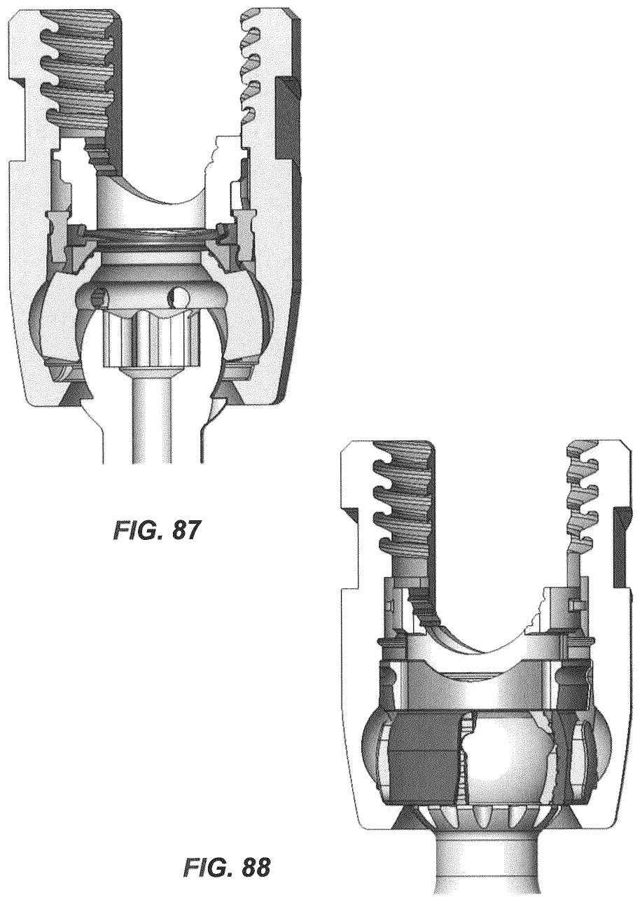

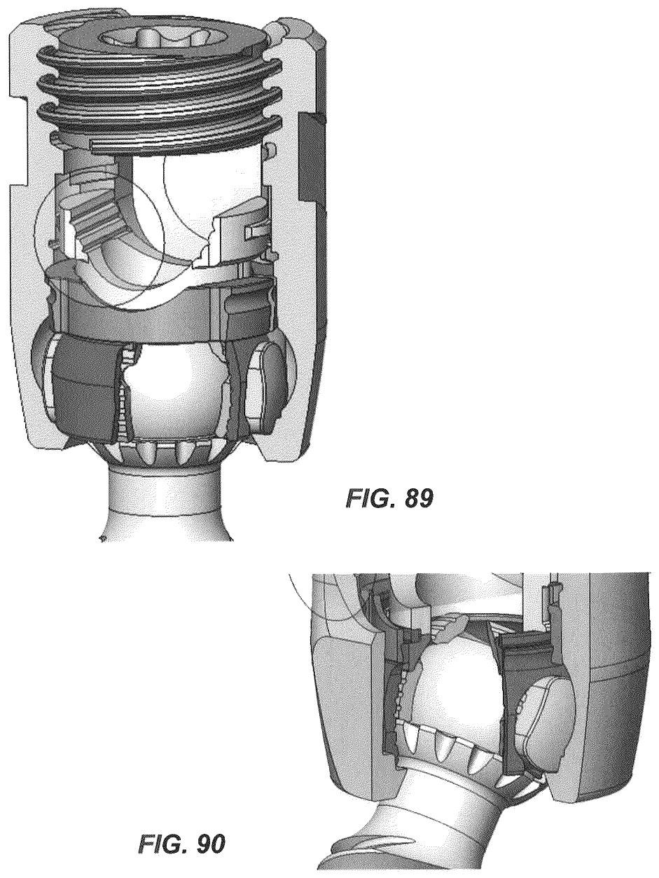

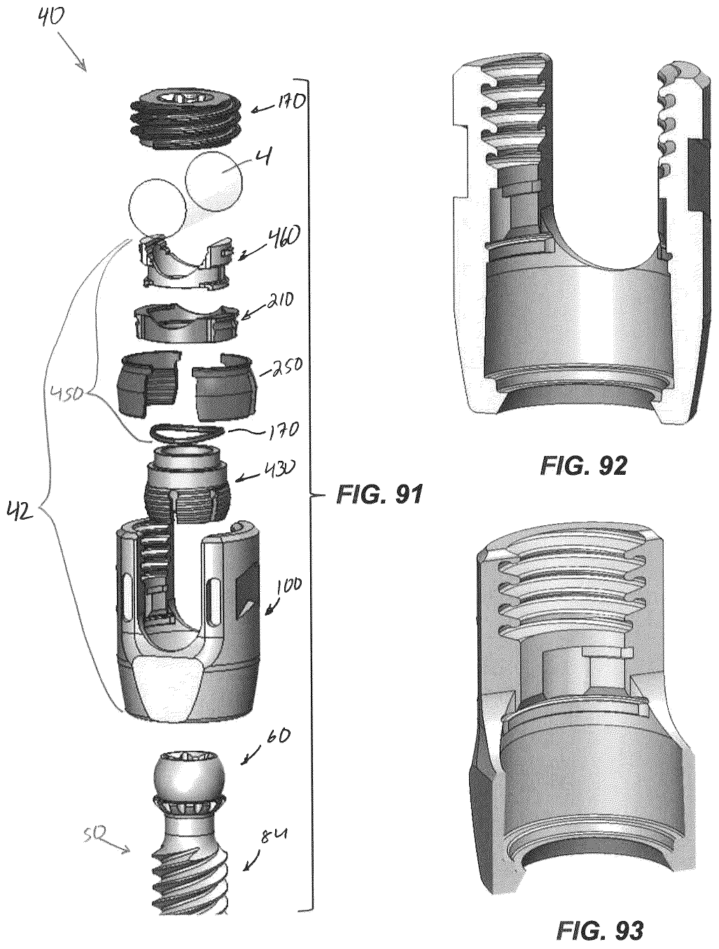

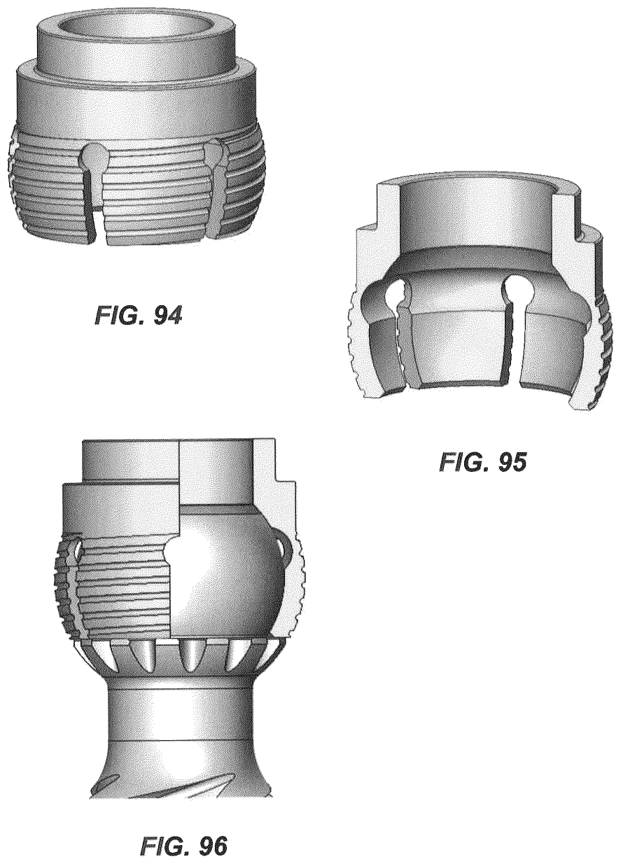

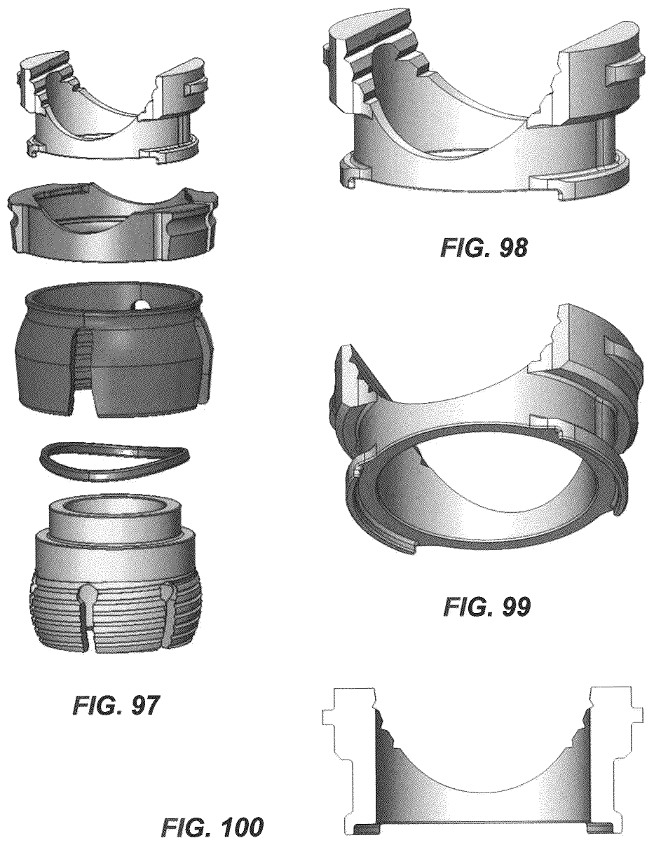

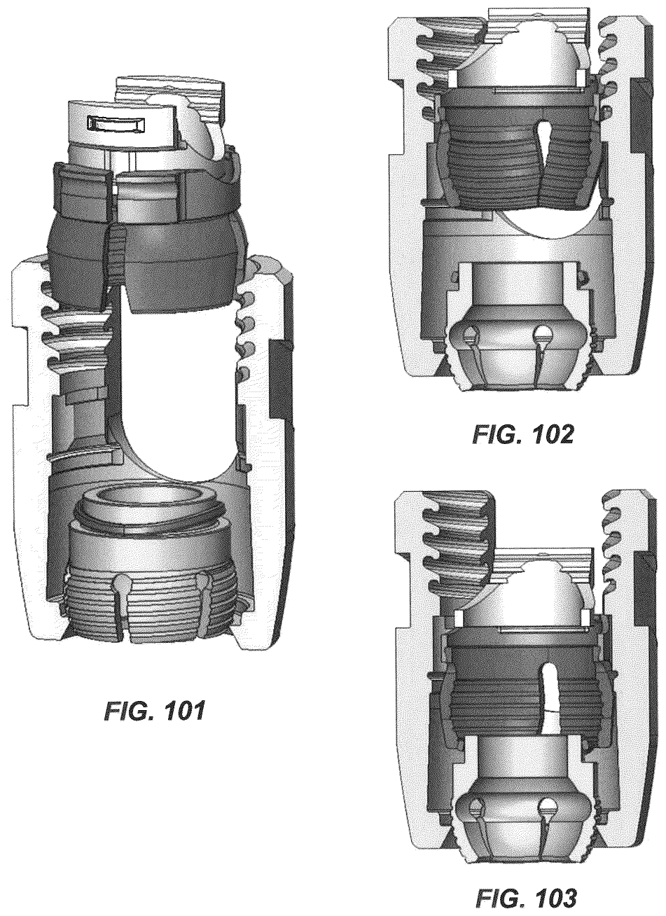

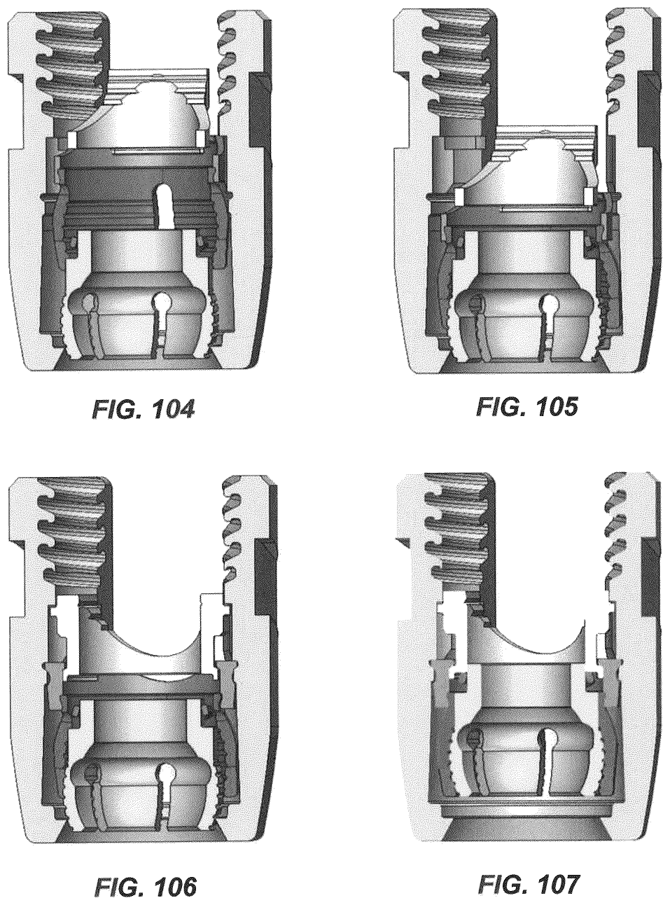

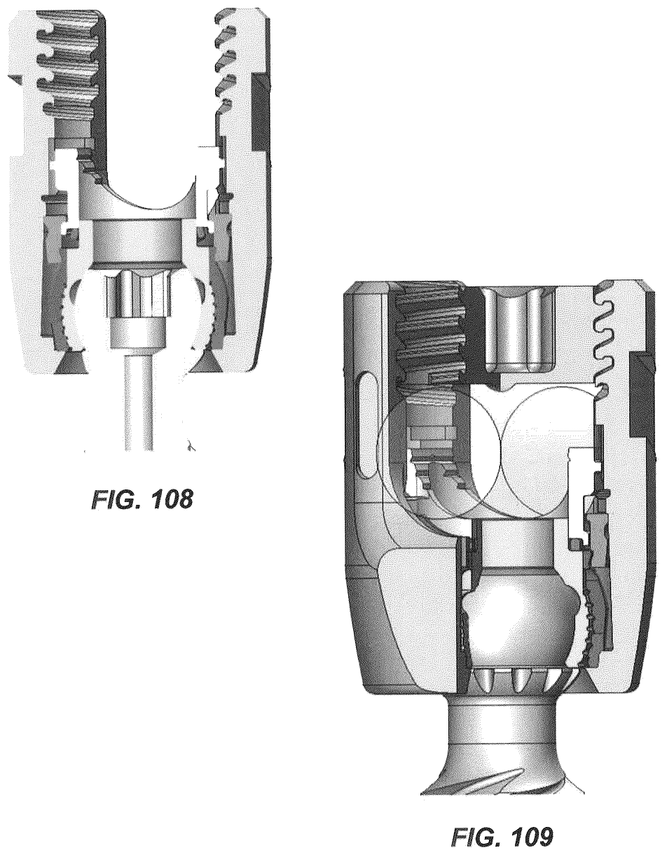

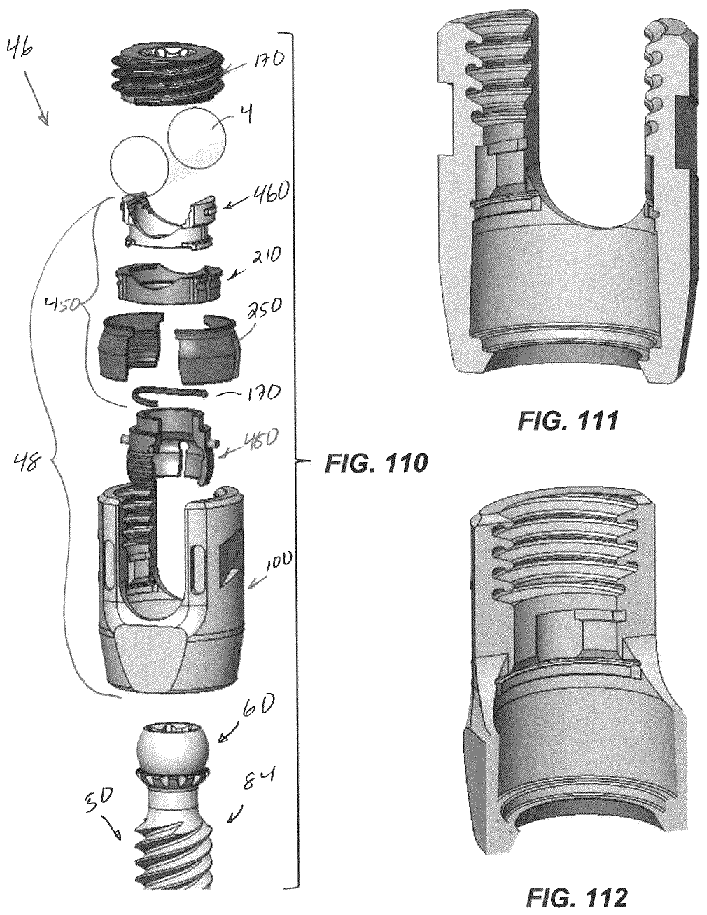

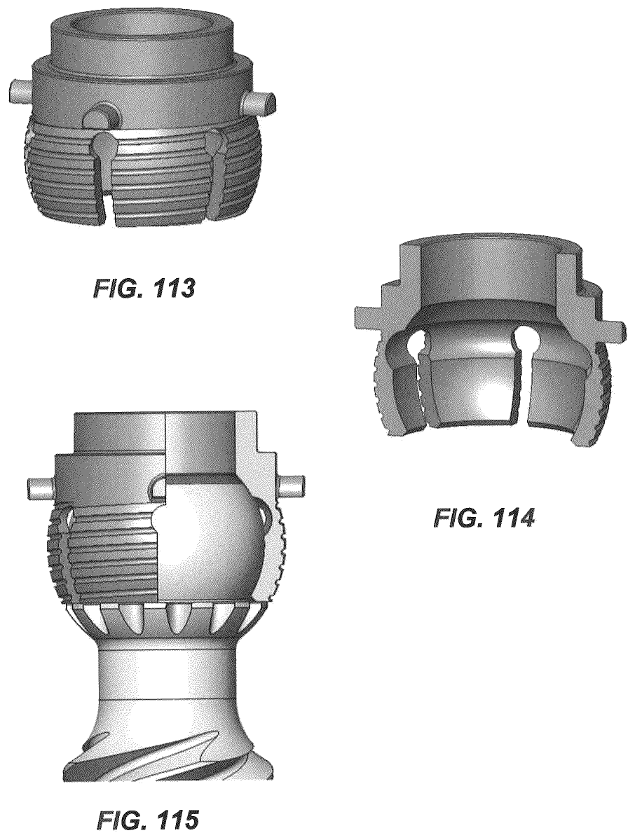

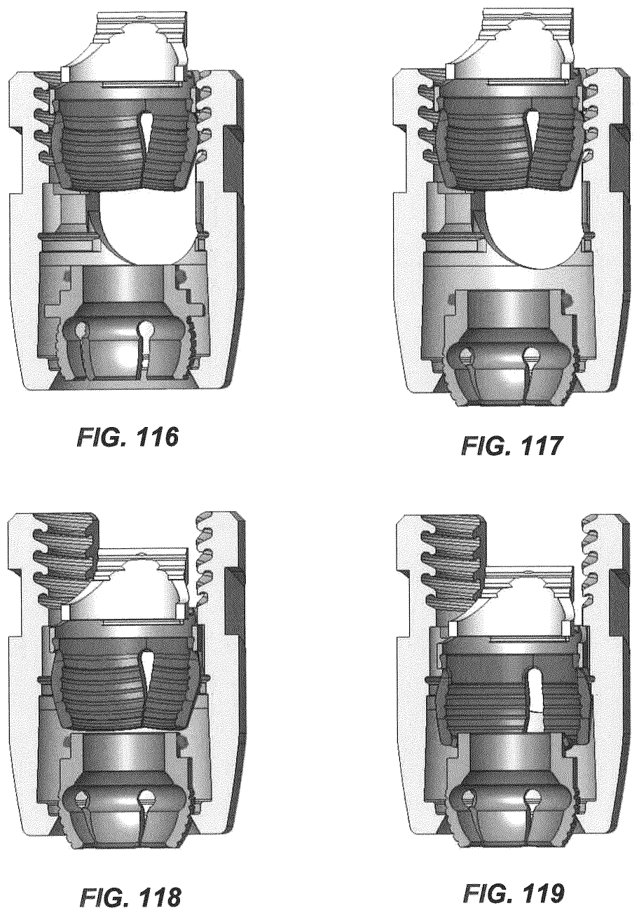

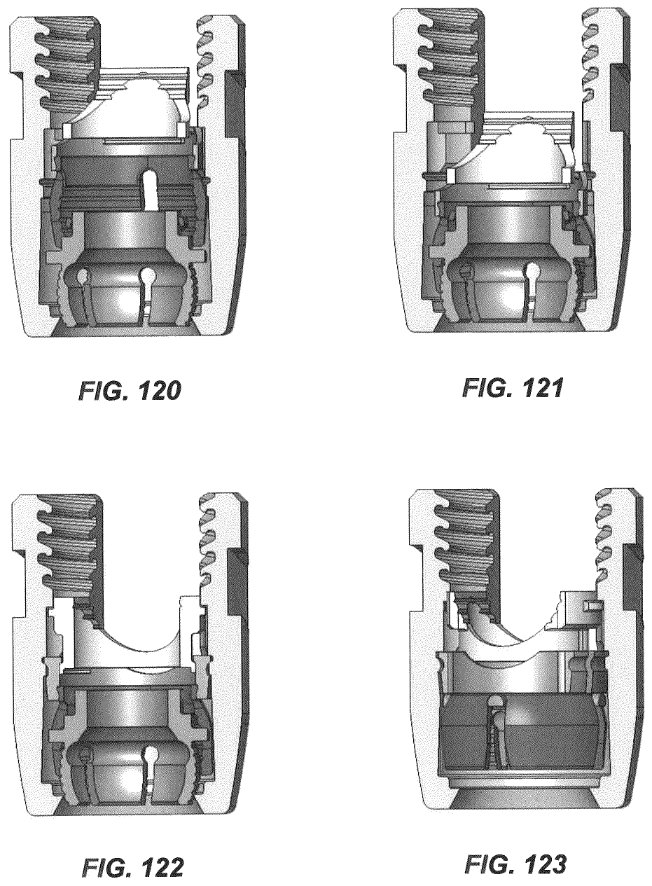

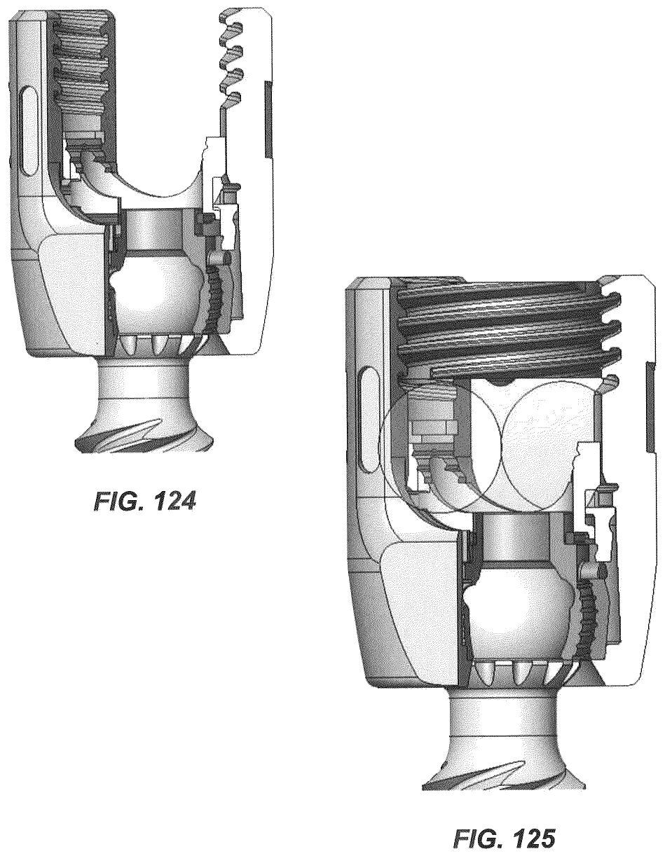

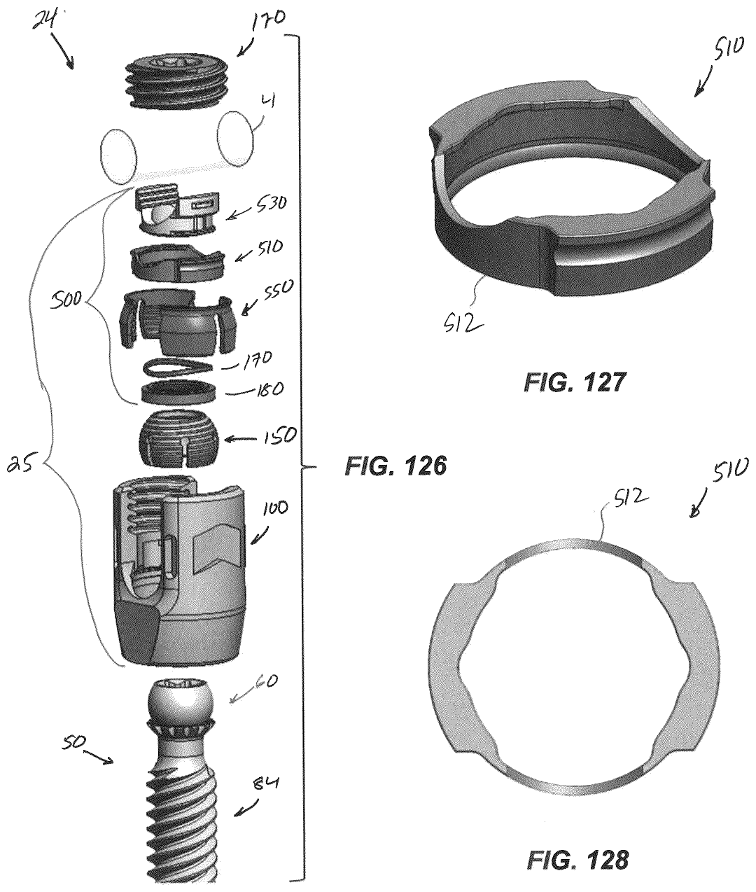

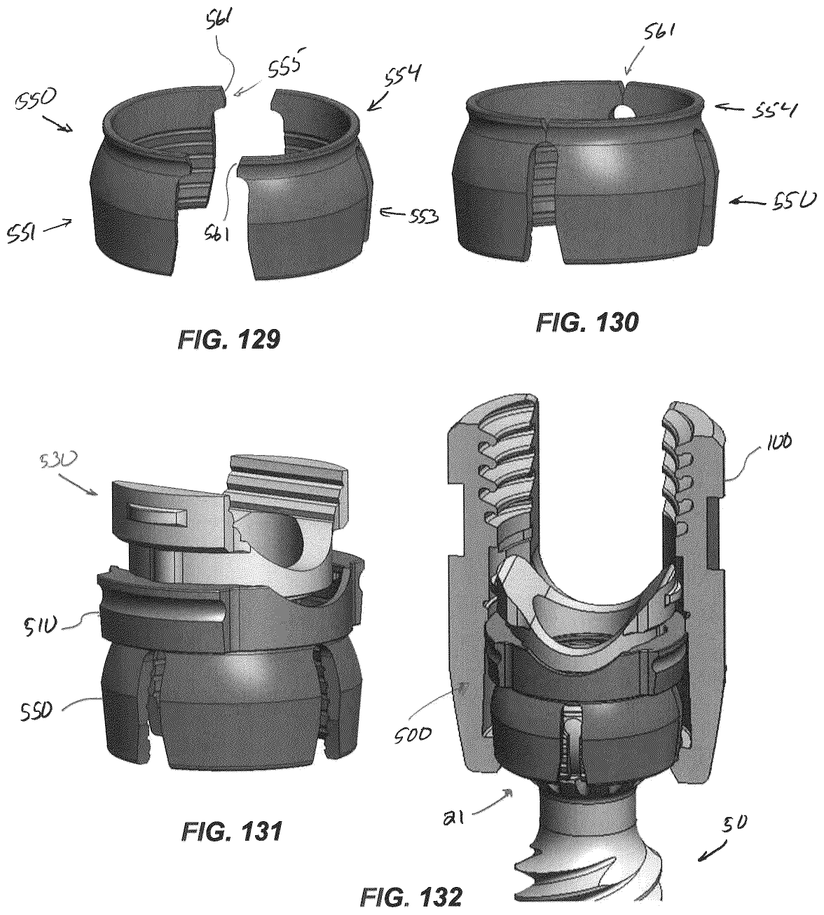

Illustrated in FIGS. 11-12 is the multiplanar embodiment of the cap retainer 150, and in FIGS. 13-14 the multiplanar cap retainer 150 coupled to the bi-spheric shank head 60. In general, the cap retainer 150 can include a partial spherical inner surface 158 configured to frictionally engage the upper partial spherical surface 66 of the bi-spheric shank head 60, and a plurality of slots 162 extending upward from a discontinuous annular bottom surface 160 to an upper solid ring portion 154 to define a plurality of flexible collet fingers 164 configured to expand to receive the bi-spheric shank head 60 when the shank head is uploaded through the lower central opening 165 of the cap retainer 150. Upon assembly together, the discontinuous annular bottom surface 160 of the cap retainer 150 can become fully engaged with the upward-facing planar ledge surface 72 of the bi-spheric shank head 60, so as to keep the cap retainer 150 from dislodging and sliding across the upper partial spherical surface 66.

In particular, and with reference to FIGS. 11-12, the multiplanar embodiment of the cap retainer 150 can have the form of a hollow, partial spherical shell with a solid or continuous upper ring portion 154 having an annular planar upper surface 152 with a continuous circular inner edge 151, and inner cylindrical surface 157 that defines a central upper opening 155. As can be seen in the drawings, a discontinuous outer spherical surface 156 extends downward from the continuous circular outer edge 153 of the upper surface 152 toward a discontinuous annular bottom surface 160, and a discontinuous inner spherical surface 158 extends downward from a lower edge of the inner cylindrical surface 157 toward the discontinuous annular bottom surface 160. In one aspect the discontinuous outer spherical surface 156 can include surface texturing or grooves, such as spiral-wound groove 168, that can improve the frictional engagement between the outer spherical surface 156 and other components in the insert sub-assembly

The distance between the discontinuous inner spherical surface 158 and the discontinuous outer spherical surface 156 can define the thickness of the partial spherical shell that forms the cap retainer. A plurality of slots 162 can be formed through the thickness of the cap retainer 150, through from the discontinuous inner spherical surface 158 to the discontinuous outer spherical surface 156 and extending upward from the discontinuous annular bottom surface 160 toward the continuous circular upper flange portion. The slots 162 can be equally spaced around the circumference of the cap retainer 150 to form a plurality of flexible collet fingers 164 extending downward from the upper ring portion, and which collet fingers 164 can flex outwardly at their lower ends, so as to expand a central lower opening 165 of the cap retainer 150 to receive the bi-spheric shank head 60 of the shank 50. It will be appreciated that the discontinuous outer spherical surface 156, the discontinuous inner spherical surface 158, and the discontinuous annular bottom surface 160 can be considered ‘discontinuous’ due to the interruptions in the surfaces created by plurality of slots extending upwardly through the lower edge and thickness of the lower portions of the shell forming the cap retainer 150, and that other terminology may also be applicable.

In addition, it is foreseen that alternative configurations, structures, or materials that provide the cap retainer with its expansion and contraction functionality are also possible and considered to fall within the scope of the present disclosure. For example, in one alternative embodiment the circular upper flange portion of the cap retainer may not be solid or continuous, and instead can include a through slot or gap extending from the discontinuous annular bottom surface or from one of the slots upwards through the upper flange portion to the annular planar upper surface 152, so as to make the cap retainer an open ring structure that is both compressible and expandable for uploading into the internal cavity of the receiver through the bottom opening.

As shown in the drawings, the cap retainer 150 can also include an inner beveled edge surfaces 159 between the discontinuous bottom annular surface 160 and the discontinuous inner spherical surface 158, which inner beveled edge surfaces 159 can define the expandable central lower opening 165 of the cap retainer. In addition, a rounded or curvate inner groove 166 can be formed into an upper portion of the discontinuous inner spherical surface 158 below the circular upper ring portion 154, which groove 166 can serve to reduce the thickness of cap retainer 150 near the roots of the downwardly-extending collet fingers 164 and thereby reduce stress in the material and facilitate the expansion of the collet fingers 164 during uploading of the bi-spheric shank head. In one aspect the upper ends of the slots 162 formed through the thickness of the cap retainer can also be formed as rounded apertures 163 or curved stress-relieving end passages.

The diameter of the discontinuous inner spherical surface 158 of the cap retainer 150 is substantially equal to the minor diameter 67 defined by the upper spherical surface 66 of the bi-spheric shank head 60 (see FIG. 4), while the diameter of the discontinuous outer spherical surface 156 is substantially equal to the major diameter 77 defined by the lower spherical surface 76 of the bi-spheric shank head 60 and an inner diameter of the clamp positioner 250, as described below. As such, the cap retainer 150 can be sized and shaped so that once positioned on the bi-spheric shank head 60, the discontinuous inner spherical surface 158 can mate or engage with the upper spherical surface 66 while the discontinuous annular bottom surface 160 engages with the lower upward-facing shelf or ledge 72 of the bi-spheric shank head. In this coupled or captured configuration, in one aspect the annular planar upper surface 152 of the cap retainer 150 can extend above the annular planar top surface 62 of the bi-spheric shank head 60, as shown in FIGS. 13-14, with the central upper opening 155 being aligned with and providing access to the drive socket 54 located immediately below. It is nevertheless foreseen that in other embodiments the top surface 62 of the bi-spheric shank head 60 can be substantially flush and aligned with, or even proud relative to, the upper surface of the cap retainer in the coupled or captured configuration. It is also foreseen that the upper surface of the cap retainer and/or the top surface of the bi-spheric shank head may not be planar.

Illustrated in FIG. 16 is a partially-sectioned perspective view of the multiplanar embodiment of the insert sub-assembly 200 as completely assembled together and coupled to the bi-spheric shank head 60 within the receiver 100 to form the multiplanar bone anchor assembly 20 shown in FIG. 1(b). FIG. 17 an exploded perspective view of the same insert sub-assembly 200 in isolation, showing again the individual components including the support collar 210, the load saddle 230, the clamp positioner 250, the wave washer 270, and the crown element 280.

Illustrated in FIG. 17 are the wave washer 270 and crown element 280 of the multiplanar insert sub-assembly, which are positionable within the central aperture of the support collar. The wave washer 270 can comprise a closed ring body 272 having an upper surface 271, a lower surface 273, a cylindrical inner surface 274 defining a center aperture 275, and a cylindrical outer surface 276. In one aspect the height of the closed ring body 272 between the upper surface 271 and the lower surface 273, taken in cross-section, can be greater than the width between the inner surface 274 and the outer surface 276. As known in the art, the closed ring body 272 can be deformed into a wave-like shape that provides a resilient biasing force when simultaneously compressed from above and below. In one aspect the upper and lower surfaces of the wave washer 270 can be reversed. Moreover, it is foreseen that other embodiments of the wave washer may also include a slit or slot to form an open ring body.

The crown element 280 can also comprise a closed ring body 282 with an inner cylindrical surface 284 defining a center opening 285 sized and shaped to receive a drive tool therethrough. The closed ring body 282 can also include a stepped upper surface configuration comprising an annular top contact surface 286, an annular top compression surface 288, and a cylindrical transition surface 287 extending between the two. The closed ring body 282 can further include a cylindrical or slightly-tapered outer surface 283 and a partial spherical downward-facing surface 289 that is sized and shape to engage the upper portions of the multiplanar cap retainer. The stepped upper surfaces 286, 287, 288 of the crown element 280 can be configured to engage and locate the wave washer 270 within the insert sub-assembly, and the partial spherical downward-facing surface 289 can be configured to transfer downwardly directed forces from insert sub-assembly or the elongate rod to the upper portions of the multiplanar cap retainer.

As discussed below, the wave washer 270 can engage a stepped bottom surface of the load saddle 230 that provides a firm support surface for the wave washer 270 to press against and apply a downwardly directed force onto the crown member 280 when compressed. In turn, the crown member can transfer the downwardly directed force to establish a frictional engagement between the partial spherical outer surface of the multiplanar cap retainer and the discontinuous curvate inner surface of the clamp positioner.

Illustrated in FIGS. 18-21 is the support collar 210, which in one aspect can function as the central base or support structure of the insert sub-assembly 200 that aligns and holds the other components together as they interact to perform the functions of the sub-assembly. The support collar 210 can comprise a substantially ring-shaped body 212 having a planar top surface 211, a planar bottom surface 213, and a cylindrical inner surface 214 defining a center aperture 215. In addition, the ring-shaped body 212 can be further separated into opposite flange sections 220 having a thickness that is greater than the thickness of the thinner cylindrical sections 218 that connect the opposite flange sections 220. The thinner cylindrical sections 218 can have partial cylindrical outer surfaces 219, as well as curvate recessed portions 217 formed into their top surfaces that, in one aspect, can facilitate the initial assembly of the load saddle to the support collar 210.

The opposite flange sections 220 can have a variety of functional features formed thereon. For example, each of the opposite flange sections 220 can include an upper outer rim 222 having an arcuate profile that extends the planar top surface 211 radially outward to a diameter that is greater than the diameter of the partial cylindrical outer surfaces 219. As described in more detail below, the opposite upper outer rims 222 are sized and shaped to rotatably slide into the shipping state grooves 130 upon assembly of the insert sub-assembly into the receiver 100. Below the upper outer rims 222 are opposite horizontal recesses 224 that, in one aspect, can have curvate profiles sized and shaped to receive the opposed horizontal ridges 132 located just below the shipping state grooves 130. Extending below the opposite horizontal recesses 224 are lower outer surfaces 225 that can taper downwardly and inwardly toward the planar bottom surface 213 of the ring-shaped body 212. As shown below, the support collar 210 is downwardly deployable within the central bore 120 of the receiver 100 until the opposite upper outer rims 222 snap under the arcuate planar underside surfaces 134 of the opposed horizontal ridges 132, which configuration can function to prevent any movement of the support collar 210 back up within the central bore.

In one aspect the support collar 210 can also include a through slot 216 that allows for expansion and/or contraction of the ring-shaped body 212. As shown in the drawings, the through slot 216 can be formed through the center of one of the opposite flange sections 220

With continued reference to FIGS. 18-21, each of the opposite flange sections 220 can also include an upper inner rim 226 that extends the planar top surface 211 radially inward. The upper inner rim 226 can have a non-arcuate profile that is shaped to interface with the outer surfaces of the load saddle, as described below. In addition, the cylindrical inner surface 214 extending downward below the upper inner rim 226 can include a shaped internal engagement groove 228 at its lower extent, with the internal engagement groove 228 having a downward-facing annular planar upper groove surface 227 and a curvate lower groove surface 229 that extends downward toward the planar bottom surface 213 of the ring-shaped body 212.

Illustrated in FIGS. 22-25 is the load saddle 230 of the multiplanar insert sub-assembly 200, in which a pair of insert arms 232 can extend upward from a ring-shaped base 240 to define an insert channel 236 configured to receive the elongate rod. In one aspect the insert channel 236 can further comprise a hybrid insert channel or saddle that is configured to receive rods of differing size. For example, in one aspect the center portion of the insert channel can have a radius of curvature configured to closely receive and center a 4.5 mm rod, while the upper outer portions of the insert channel can have a radius of curvature configured to closely receive and center a 5.0 mm rod, as illustrated in FIGS. 62-63. Other configurations for the different radii of curvature, such as for larger or smaller rods, are also contemplated and configured to fall within the scope of the present disclosure.

The insert arms 232 of the load saddle 230 can include opposite upper flange sections 234 having a thickness that is greater than the thickness of the ring-shaped base 240. The upper flange sections 234 can have planar top surfaces 233 and partial cylindrical outer side surfaces 235 with an outer diameter sized to be closely received within the upper discontinuous cylindrical surface 124 of the receiver 100. As previously described, the load saddle 230 can also include indexing nubs 237 projecting outwardly from a center portion of the outer side surfaces 235 of the upper flange sections 234, which indexing nubs 237 are sized and shaped to be initially received within the horizontal access recesses 127 during pre-assembly of the insert sub-assembly with the receiver 100, and then further received within the vertically-elongate side pockets 126 upon completion of the pre-assembly.

With continued reference to FIGS. 22-25, the ring-shaped base 240 of the load saddle 230 can include an inner cylindrical surface 242 defining a center opening 243 sized and shaped to receive a drive tool therethrough, and an outer cylindrical surface 244. The ring-shaped base 240 of the load saddle can further includes a pair of opposite lower flanges 246 projecting radially outward below the opposite upper flange sections 234. The opposite lower flanges 246 can have outer edge surfaces 247 configured to engage the cylindrical inner surface 214 of the support collar 210, and upper surfaces 245 configured to engage the underside surfaces of the upper inner rims 226. In addition, opposite vertical alignment rails or bars 238 can be formed with and extend upward from a center portion of the opposite lower flanges 246 to lower surfaces of the opposite upper flange sections 234. The opposite vertical alignment bars 238 can have outer profiles that are sized and shaped for slidable engagement with the center portions of the inner edge surfaces of the upper inner rim 226 of the support collar 210. As shown below, these engagements can serve together to both hold the position and control the movement of the load saddle 230 within the central aperture 215 of the support collar 210 at various times during the pre-assembly, deployment, and use of the multiplanar bone anchor assembly 20.

The ring-shaped base 240 can also include a stepped bottom surface configuration, comprising an annular bottom contact surface 248 and an annular bottom compression surface 249, that mirrors the stepped upper surface configuration of the crown element 280. As previously described, the stepped configurations can together serve to engage and locate the wave washer 270 within the insert sub-assembly 200. In addition, and as described in more detail below (see FIG. 61), the annular bottom contact surface 248 of the load saddle 230 can directly engage the annular top contact surface 286 of the crown element 280 as the completed multiplanar bone anchor assembly 20 is placed in the hard locked configuration, so as to directly transfer load from the elongate rod, through the load saddle 220 and crown element 280, to the upper surface portions of the cap retainer 150 that is coupled to the bi-spheric shank head 60.

Illustrated in FIGS. 26-29 is the two-piece clamp positioner 250 of the multiplanar insert sub-assembly 200. As shown, the clamp positioner 250 can comprise two semi-cylindrical shell-like pieces 251, 253 that can be substantially identical to each other. In particular, both pieces 251, 253 can include semi-circular upper connection portions 254 having annular planar top surfaces 252, cylindrical inner surfaces 256 that align together to define an upper opening 257, planar end surfaces 255, and a shaped outer lip 258 that mirrors the shaped internal engagement groove 228 of the support collar 210. This can include, for example, outer portions of the top surfaces 252 that engage with the downward-facing upper groove surface 227 of the internal engagement groove 228, as well as curvate outer groove surfaces 259 that closely receive the curvate lower groove surface 229.

Extending downward from the upper connection portions 254 are curvate clamp panels 260 having outer surfaces 264 and spherical inner surfaces 262 configured to receive, clamp around, and secure the discontinuous outer spherical surface 156 of the cap retainer 150 within the receiver 100. As shown, each of the upper connection portions 254 can include two clamp panels 260, providing the clamp positioner 250 with four clamp panels 260 circumferentially-spaced around the lower portion of the clamp positioner 250 for engaging the cap retainer 150. It is foreseen that other configurations for the clamp positioner are also possible (e.g., clamp positioners formed with a different number of pieces, or individual pieces having different a different number of downward extending clamp panels, and the like) and considered to fall within the scope of the present disclosure. As with the cap retainer, moreover, the spherical inner surfaces 262 of the clamp panels 260 can also include surface texturing or grooves, such as parallel grooves 169, that can improve the frictional engagement between the spherical inner surfaces 262 of the clamp positioner 250 and the discontinuous outer spherical surface 156 of the cap retainer 150.

The clamp panels can be separated by vertical slots 266 extending upward from the bottom edge of the clamp positioner 250, with the arcuate planar bottom surfaces of the four clamp panels 260 combining to define a discontinuous annular bottom surface 268. As described below, the lower ends of the clamp panels 260 can eventually be received within the annular locking recess 142 of the receiver, with the discontinuous annular bottom surface 268 being engaged with the annular, upward-facing lower step surface 144 and the lower portions of the outer surfaces 264 of the clamp panels 260 being engaged with the lower sidewall surface 143.

Illustrated in FIGS. 30-37 is the pre-assembly of the multiplanar insert sub-assembly 200 prior to downloading the complete sub-assembly into the receiver. With reference to FIGS. 31-33, for instance, the base 240 of the load saddle 230 can be downloaded into the central aperture 215 of the support collar 210 in a non-aligned position (FIG. 31), until the upper surfaces 247 of the lower flanges 246 of the load saddle 230 are located below the level of the upper inner rims 226 of the support collar 210 (FIG. 32). The load saddle 230 can then be rotated relative to the support collar 210, with the lower flanges 246 rotating under the upper inner rim 226 and the outer edges 247 of the lower flanges 246 sliding along the cylindrical inner surface 214. Around mid-quarter turn the opposite vertical alignment bars 238 can begin to engage the inner edges of the upper inner rim 226, causing the open ring body 212 of the support collar 230 to flex open at the slot 216. The load saddle 230 can continue to rotate a full quarter turn or 90 degrees until the opposite vertical alignment bars 238 reach the complementary shaped recesses formed into the center portions of the upper inner rims 226, allowing the open ring body 212 to closes at the slot 216 to capture the load saddle 230 to the support collar 210. (FIGS. 32-33). In this position the insert arms 232 and the indexing nubs 237 are aligned with the opposite flange sections 220 of the support collar 210.

With reference to FIGS. 34-35, the upper connection portions 254 of the clamp positioner 250 can then be uploaded into the central aperture 215 of the support collar 210 below the load saddle 230, such that the shaped outer lips 258 and outer groove surfaces 259 of the two pieces 251, 253 forming the clamp positioner 250 are closely received with the shaped engagement groove 228 and lower groove surface 229 formed into the cylindrical inner surface 214 of the support collar 210. In one aspect the open ring body 212 of the support collar 230 can be expanded to accommodate the uploading of the two-piece clamp positioner 250 as a single body. However, it is foreseen that alternative structures and other methods for uploading of the clamp positioner are also possible and considered to fall within the scope of the present disclosure.

With reference to FIGS. 36-37, the wave washer 270 and the crown element 280 can be uploaded into the central aperture 215 of the support collar 210, with the wave washer 270 entering into simultaneous compressive engagement with the annular bottom compression surface 249 of the load saddle 230 and the annular top compression surface 288 of the crown element 280. This can serve to create a pre-tenson between the several components that can hold the insert sub-assembly 200 together. It will be appreciated that the positioning and engagement of the solid ring crown element 280 against the cylindrical inner surfaces 256 of the upper connection portion 254 of the clamp positioner 250 can serve to prevent the support collar 210 from contracting during subsequent positioning and deployment of the insert sub-assembly within the receiver. In one aspect the support collar 210 can be resiliently expanded by the uploading of the crown element 280 into the clamp positioner 250, which resilient expansion can create a pre-tension that holds the insert sub-assembly together. In another aspect, either the cylindrical outer surface 283 of the crown element 280 or the cylindrical inner surface 256 of the upper connection portion 254 of the clamp positioner 250 could be formed with a slight taper that establishes an interference compression fit to create the pre-tension forces.

Illustrated in FIGS. 38-48 is the pre-assembly of the cap retainer 150 and insert sub-assembly 200 into the receiver 100 to form the multiplanar receiver sub-assembly 22 in the shipping state position or configuration. With reference to FIG. 38, the multiplanar cap retainer 150 is first downloaded into the internal cavity 136 of the receiver of the receiver 100, with the lower portions of the discontinuous outer spherical surface 156 of the cap retainer 150 resting on the circular edge 146 that defines the bottom opening 145. The insert sub-assembly is then positioned over the central bore 120 of the receiver 100 with the flange sections 220 of the support collar 110 and the insert arms 232 of the load saddle 230 being aligned with the open channel 106 of the receiver 100, and with the insert channel 236 of the load saddle 230 being non-aligned with or transverse to the open channel 106.

With reference to FIGS. 39-42, the insert sub-assembly 200 can now be downloaded into the receiver 100. As shown in FIGS. 39-40, this can be accomplished by compressing the lower ends of the curvate clamp panels 260 of the clamp positioner 250 together along the split line between the two pieces 251, 253, which can reduce the maximum width between of the outer surfaces 264 of the opposed clamp panels 260 to less than the narrowest diameter of the central bore 120, as measured between the crests of the helically-wound interlocking flange form 123 and/or the diameter of the upper discontinuous cylindrical surface 124. The insert sub-assembly 200 can then pass downward through the channel portion of the central bore, until the curvate clamp panels 260 of the clamp positioner 250 enter the internal cavity 136, as shown in FIG. 40. The lower ends of the curvate clamp panels 260 can then be expanded back to their nominal positions (FIG. 41) so that the insert sub-assembly can be further downloaded through the central bore until the support collar 210 reaches the level of the opposed shipping state grooves 130 (FIG. 42). At this point, the inner edges of the discontinuous annular bottom surface 268 of the clamp positioner 250 can contact or rest lightly on the discontinuous outer spherical surface 156 of the cap retainer 150.

With reference to FIGS. 43-45, the entire insert sub-assembly 200 can then be rotated about the vertical centerline axis of the receiver until the upper outer rims 222 of the support collar 210 first enter the opposed shipping state grooves 130 of the receiver, as shown in FIG. 43. With continued rotation toward 90 degrees, the index nubs 237 of the load saddle 230 can then enter the access recesses 127 formed into the upper discontinuous cylindrical surface 124 (FIG. 44), until eventually the indexing nubs 237 slide into the vertical side pockets 126 and the upper outer rims 222 of the support collar 210 are fully received within the opposed shipping state grooves 130 of the receiver 100 (FIG. 45). The insert channel 236 of the load saddle 230 is now aligned with the open channel 106 of the receiver. In one aspect the slightly reduced or shallower depth of the access recesses 127 adjacent the vertical side pockets 126, as described above, can create some resistance to the rotation of the insert sub-assembly 200 toward the end of its rotation, so that the releasing of the indexing nubs 237 as they pass completely through the horizontal access recesses 127 and enter the vertical side pockets 126 can result in a ‘snap-in’ action which confirms that the insert sub-assembly 200 is properly positioned within the receiver 100.

With reference to FIGS. 46-48, the multiplanar cap retainer 150 can then be uploaded through the lower opening of the clamp positioner 250 defined by the discontinuous annular bottom surface 268, with the curvate clamp panels 260 flexing open within the upper expansion portion of the internal cavity 136 of the receiver 100, until the cap retainer 150 is captured by the clamp positioner 280 in a stabilized position that is suspended and centered over the bottom opening 145. Accordingly, the pre-assembly together of the multiplanar versions of the receiver 100, the cap retainer 150, and the insert sub-assembly 200 to form the multiplanar receiver sub-assembly 22, in which insert sub-assembly 200 is secured in an aligned position within the open channel 106 and the cap retainer 150 is stabilized and centralized above the bottom opening 145, is now complete.

In one aspect the pre-assembly of the separate components into the multiplanar receiver sub-assembly 22, generally completed at the factory or manufacturing facility of the spine company, can be defined as the shipping state configuration of the ‘modular’ receiver sub-assembly 22, as described herein and commonly understood in the art. For example, in this configuration the multiplanar receiver sub-assembly 22 is now ready for storage and/or shipping and handling, and for eventual attachment to the bi-spheric shank head 60 of a bone anchor or shank 50 either prior to or during spinal surgery. Nevertheless, it will also be appreciated that in other embodiments the shipping state configuration can include the additional assembly of the multi-planar receiver sub-assembly 22 together with the bone anchor or shank 50 at the factory or manufacturing facility, with the pre-assembled multiplanar bone anchor assembly 20 then being shipped in trays, generally together with the closures 170, 180, to the hospital or surgery center. It will also be appreciated that in yet other embodiments the individual components described above can also be pre-assembled into the receiver sub-assembly 22 and/or bone anchor assembly 20 at the hospital or surgery center prior to implantation in a patient.

One representative embodiment or method of assembling the multiplanar receiver sub-assembly 22 to the bi-spheric shank head 60 of the bone anchor or shank 50 is illustrated in FIGS. 49-56. For instance, and with initial reference to FIG. 49, the receiver sub-assembly 22 can be first positioned above the proximal end 52 of the bone anchor 50 with the expandable central lower opening 165 of the cap retainer 150, which is stabilized and centered above the bottom opening 145 of the receiver 100 by the clamp positioner 250, being generally aligned with the upper partial spherical portion 64 of the bi-spheric shank head 60. The receiver sub-assembly 22 is then dropped downward (or the bone anchor is moved upward, depending on the frame of reference of the reader) until the upper spherical surface 66 of the bi-spheric shank head 60 passes upward through the bottom opening 145 of the receiver 100 toward the central lower opening 165.

As shown in FIG. 50, the receiver sub-assembly 22 continues to move downward (or the bone anchor moves upward) as the bi-spheric shank head 60 begins to push against the beveled edge surfaces 159 of the collet fingers 164 of the cap retainer 150 that is held in space within the internal cavity 136 by the clamp positioner 250. The upper ring portion 254 of the clamp positioner, in turn, is secured and supported within the internal cavity 136 by the support collar 210, which itself is upwardly immovable due to its engagement with the receiver 100 (via the opposite upper outer rims 222 of the support collar 210 being fully received within the opposed shipping state grooves 130). Due to this series of direct rigid engagements, the cap retainer 150 does not move upward and instead the central lower opening 165 of the cap retainer 150 can expand as both the collet fingers 164 of the cap retainer 150 and the curvate clamp panels 260 of the clamp positioner 250 are pushed apart by the upwardly-moving bi-spheric shank head 60. At the same time, the inner beveled edge surfaces 159 of the collet fingers 164 can scrape downwards across the upper spherical surface 66 of the bi-spheric shank head 60, pushing any bone debris and/or soft tissue located on the outer surface downwards before them.

The collet fingers 164 and the curvate clamp panels 260 continue to be expanded within the upper expansion portion of the internal cavity 136 by the upward movement of the bi-spheric shank head 60 into the receiver 100. The expansion of the cap retainer 150 can continue, with the inner beveled edge surfaces 159 and/or the discontinuous annular bottom surface 160 pushing any bone debris and/or soft tissue located on the upper spherical surface 66 downwards before it, until the discontinuous annular bottom surface 160 reaches the level of the hemisphere plane 65 of the bi-spheric shank head 60 and the collet fingers 164 of the cap retainer 150 are at their point of maximum expansion, as shown in FIG. 50.

With reference to FIGS. 51-53, the receiver sub-assembly 22 continues to move downward (or the bone anchor moves upward) as the upper partial spherical portion 64 of the bi-spheric shank head 60 becomes fully captured by the cap retainer 150 as it contracts to close around the upper spherical surface 66. During this motion, the discontinuous annular bottom surface 160 continues to push any bone debris and/or soft tissue downward toward the annular lower ledge 70 as the lower partial spherical portion 74 now moves upward into and through the bottom opening 145 of the receiver 100. Any bone debris and/or soft tissue that has been removed from the upper spherical surface 66 of the bi-spheric shank head 60 can pass through the plurality of open, vertically aligned flutes 78 that extend downwardly through and below the lower ledge 70 as the discontinuous annular bottom surface 160 engages the upward-facing planar surface 72 of the lower ledge 70.

The discontinuous inner spherical surface 158 of the cap retainer 150 is now secured around the upper partial spherical portion 64 of the bi-spheric shank head 60. Furthermore, with the simultaneous engagement of the discontinuous bottom annular surface 160 against the lower ledge 70 of the lower partial spherical portion 74, the cap retainer 150 is also now aligned on the bi-spheric shank head 60 so that the inner cylindrical surface 157 that defines the central upper opening 155 can be aligned with the internal drive feature or drive socket 54 of the bi-spheric shank head 60. In addition, the discontinuous outer spherical surface 156 of the cap retainer 150 can also be aligned with the lower spherical surface 76 of the lower partial spherical portion 74 so as to create a single diameter, articulating, multiplanar shank head sub-assembly 21 having the major diameter 77 that is greater than the diameter of the bottom opening 145 of the receiver 100, thereby preventing the bottom loaded shank 50 from exiting the receiver 100 back out through the same bottom opening 145 through which it was initially loaded. It will be appreciated that the cap retainer 150 can still remain a member of the receiver sub-assembly 22 even after its coupling to the bi-spheric shank head 60 to form the shank head sub-assembly 21, and as such may be considered the linking mechanism that connects the two sub-assemblies together.

With the shank head sub-assembly 21 secured within the clamp positioner 250, the multiplanar insert sub-assembly 22 can then be downwardly deployed with a deployment tool, as shown in FIGS. 53-56. With reference to FIG. 54, in one aspect the deployment tool 6 can include a rounded lower surface 7 that is complementary with the upward-facing surface of the insert channel of the load saddle. However, the primary driving engagement can generated by end effectors 8 that extend around and below the rounded lower surface 8 to engage the top surfaces of the cylindrical sections 218 of the support collar 210. As shown, the end effectors 9 can include curvate engagement surfaces 9 that are complementary with the curvate recessed portions 217 of the cylindrical sections 218. It is foreseen that a variety of other structural features for providing contact engagement between the deployment tool and the support collar are also possible and considered to fall within the scope of the present disclosure.

With continued reference to FIG. 55, the deployment tool 6 can be used to drive the support collar 210 downward within the central bore 120, which can push the underside surfaces of the upper outer rims 222 of the support collar 210 downward along the ramped lower surfaces 131 of the shipping state grooves 130 until the upper outer rims 222 reach and scrape across the opposed horizontal ridges 132, after which the upper outer rims 222 snap under the arcuate planar underside surfaces 134 of the opposed horizontal ridges 132. With the same motion the clamp positioner 250, which is rigidly coupled to the support collar 210, is also driven downward until the lower ends of the clamp panels 260 become engaged within the annular locking recess 142 of the receiver 100, with the discontinuous annular bottom surface 268 being engaged with the annular, upward-facing lower step surface 144 and the lower portions of the clamp panels 260 being engaged with the lower sidewall surface 143. It will be appreciated that the timing of the connections between the upper outer rims 222 with the planar underside surfaces 134 of the receiver 100, and between the lower portions of the clamp panels 260 and the annular locking recess 142, can be substantially simultaneous. Upon completion of the deployment and removal of the deployment tool 6, as shown in FIG. 56, the upper and lower connections can serve to rigidly secure the support collar 210 and the clamp positioner 250 as a unit to the internal structures of the central bore 120, and thereafter prevent these outer components of the insert sub-assembly 200 from moving back up (or down) within the receiver 100.

The coupling of the universal bi-spheric shank head 60 of the bone anchor or shank 50 with the multiplanar receiver sub-assembly 22 can complete the formation of the multiplanar bone anchor assembly 20 in its initial configuration, one in which the multiplanar bone anchor assembly 20 is ready to be implanted into the vertebrae of a patient or to receive the elongate rod and the closure.

FIGS. 58-59 provide cut-away views of the multiplanar bone anchor assembly 20 upon the initial assembly of the multiplanar receiver sub-assembly 22 to the bi-spheric shank head 60, but prior to final assembly with the elongate rod and closure top. In this configuration the articulating multiplanar shank head sub-assembly 21 (i.e. the multiplanar cap retainer 150 and the bi-spheric shank head 60) can be secured against the discontinuous spherical inner surfaces 262 of the two-piece clamp positioner 250 with a frictional engagement, or pre-lock friction fit, that allows for the bone anchor or shank 50 to both pivot and rotate relative to the receiver 100 as the outer surfaces of the shank head sub-assembly 21 (i.e., the discontinuous outer spherical surface 156 of the cap retainer 150 and the lower spherical surface 76 of the bi-spheric shank head 60) slidably frictionally engage with the discontinuous spherical inner surfaces 262 of the clamp positioner 250 with a ball and socket-type connection.

In one aspect this friction fit can be provided by the wave washer 270 that is located between the load saddle 230 and the crown element 280. In particular, with the load saddle 230 being held in a vertical position by the engagement between the upper surfaces 245 of the opposite lower flanges 246 and the underside surfaces of the upper inner rims 226 of the support collar 210, the annular bottom compression surface 249 of the load saddle 230 can provide a firm support surface for the wave washer 270 to press downward upon the annular top compression surface 288 of the crown element 280. The crown element 280, in turn, has a partial spherical downward-facing surface 289 configured to apply the downwardly directed force to upper portions of the discontinuous outer spherical surface 156 of the cap retainer 150, thereby creating the frictional engagement between the cap retainer 150 and the clamp positioner 150.

With reference to FIGS. 59-60, in one embodiment the closure 170 can comprise a single-piece closure having a generally cylindrical closure body 172 with a top surface 171, a bottom surface 173, and an outer continuous guide and advancement structure 177 formed into the outer side surface 176 of the closure body 172 that operably joins with the discontinuous guide and advancement structure 122 formed into the upright arms 210 of the receiver 100. As illustrated, the outer continuous guide and advancement structure 176 can be a dual flange/dual lead-in guide and advancement structure having first and second helically wound interlocking closure flange forms 178 and corresponding first and second starts 179. In one aspect the closure flange form 178 can include a splay-resisting or splay-controlling flange profile for operably guiding under rotation and advancing the closure 170 downward between the upright arms and having such a nature so as to resist, inhibit, limit, or preferentially control the splaying of the upright arms when the closure is advanced into the rod channel. In other embodiments not shown, the outer continuous guide and advancement structure can be a single flange/single lead-in guide and advancement structure having a single closure flange form and corresponding single start, or may have more than two closure flange forms and corresponding starts. In other aspects, the guide and advancement structure may take on a variety of alternative forms, including but not limited to single closure thread/single start, dual closure threads/dual starts, buttress threads, square threads, reverse angle threads, interlocking gripping or dovetail-like threads, or other thread-like or non-thread-like helically wound advancement structures.

As shown in the drawings, in one aspect the bottom surface 173 of the closure 170 can include a downwardly-projecting central point 175 for engaging and securing the elongate rod. In other embodiments the bottom surface can include an annular projection, a point ring (i.e., an annular ring surrounding a central point or projection), a downwardly-projecting stepped planar surface for controlling the closure torque to thrust ratio, a recessed surface surrounded by a low outer ridge, and the like. In yet other embodiments the bottom surface can be substantially planar across the extent thereof. In yet other embodiments the closure can have a through-and-through central opening.

The top surface 171 of the closure 170 can further include a driving tool engagement structure, such as a central internal drive socket 174, which extends downward or inward into the body of the closure. The internal drive socket 174 can be used for closure installation or removal. Similar to the internal drive feature formed into the shank head, the internal drive socket of the illustrated closure is an aperture formed in the top surface, and in one aspect can be a multi-lobular or star-shaped aperture, such as those sold under the trademark TORX, or the like, having internal faces designed to receive a multi-lobular or star-shaped tool for rotating and driving the closure. It is foreseen that such a driving tool engagement structure may take a variety of tool-engaging forms and may include one or more apertures of various shapes, such as a plurality of bores of different diameters, a pair of spaced apart apertures or a hex shape designed to receive a hex tool (not shown) of an Allen wrench type. In one aspect the seat or base surface of the internal drive socket can be disposed perpendicular to a closure axis, with the internal drive socket otherwise being coaxial with the axis. In yet other embodiments the internal drive socket can extend entirely through the closure.

In another aspect of the present disclosure, a break-off extension (not shown) can be attached the upper end or top surface of the closure, and extend upwardly away therefrom to provide an external tool engagement structure that can be used for rotatably advancing the closure downward between the upright arms of the receiver. In one aspect the break-off extension can be designed to allow the extension to break from the closure at a preselected torque, for example, 60 to 140 inch pounds. It is further foreseen that closures having other shapes, configurations, thread forms or non-threaded engagement alternatives, and the like, that are different from those shown in the drawings while providing for similar interaction and functionality of the various components of the bone anchor assembly, are also possible and considered to fall within the scope of the present disclosure. For example, the thread forms can have a thread depth, as measured from its root to its crest, which is equal to half or less than half of the thread pitch.

Illustrated in FIG. 61 is the multiplanar bone anchor assembly 20 after final assembly with the elongate rod 4 and the single piece closure 170, and in FIGS. 63-64 with differently-sized elongate rods 4a, 4b being positioned within the insert channel 236 of the load saddle 230, as discussed above. In these configurations the load saddle 230 can be pressed downward by the elongate rod 4 and closure 170 against the biasing force of the wave washer 270, until the annular bottom contact surface 248 of the load saddle 230 directly engages the annular top contact surface 286 of the crown element 280 to hard or fully lock the discontinuous outer spherical surface 156 of the cap retainer 150 against the discontinuous spherical inner surfaces 262 of the clamp positioner 250. In one aspect the wave washer 270 can also be fully compressed between the annular bottom compression surface 249 of the load saddle 230 and the annular top compression surface 288 of the crown element 280 in the hard locked configuration.

Finally, it will be appreciated that subsequent limited unthreading or backing-off of the single piece closure 170 from the receiver 100, without removing the elongate rod 4 or completely detaching the closure 170, can allow the wave washer 270 to press the load saddle 230 back upward within the rod channel 106 to release the hard lock of the multiplanar bone anchor assembly 20 and re-establish the non-floppy, friction fit configuration between the components of the multiplanar bone anchor assembly 20. A slight wiggling of the multiplanar receiver sub-assembly 22 can then serve to re-mobilize the multiplanar receiver sub-assembly 22 relative to the bi-spheric shank head 60 and allow its position to be adjusted prior to re-locking the multiplanar bone anchor assembly 20 in a new position with a hard lock using the closure 170.

### Independent Lock Bone Anchor Assembly

Referring now to FIG. 66, illustrated therein is an exploded perspective view of the representative embodiment of the multiplanar IL bone anchor assembly 26 is illustrated in FIG. 1(c). In addition to the features and functions of the previous multiplanar embodiment, the multiplanar IL bone anchor assembly 26 is configured to provide an independent lock (“IL”) functionality in that the position of the IL receiver sub-assembly 28 can be immovably hard locked to the bi-spheric shank head 60 of the bone anchor or shank 50 independent of the elongate rod 4 being locked within the rod channel of the receiver. This multiplanar IL bone anchor assembly 26 can include the same bone anchor, having a bi-spheric shank head 60 and an anchor portion 84 opposite the bi-spheric shank head 60 for securement or attachment to the bone of a patient.

The multiplanar IL bone anchor assembly 26 also includes a multiplanar receiver 100 that can be initially pivotally secured to the bi-spheric shank head 60 with many of the same internal components that have been pre-assembled into the internal cavity 136 and the rod channel 106 of the receiver to form the multiplanar IL receiver sub-assembly 28, including the pivoting or articulating multiplanar cap retainer 150. These internal components can further include the multiplanar IL insert sub-assembly 204, in which the upwardly-extending insert arms 292 of the IL load saddle 290 have been lengthened to extend upward above the top of the elongate rod 4 when it is positioned within the open insert channel. Before or after the elongate rod 4 has been positioned within the insert channel 293 and the lower portion of the rod channel 106, a two-piece closure 180 can be threadably or otherwise secured into the upper portion of the rod channel 106 to separately apply pressure to upper surfaces 291 of the insert arms 292 and to the upper surface 3 of the elongate rod 4, eventually locking both the elongate rod 4 and the IL receiver sub-assembly 28 into a final or hard locked position relative to the bone anchor or shank 50.

The primary difference between the multiplanar IL bone anchor assembly 26 and the previous embodiment can be the alternative IL load saddle 290 and the alternative two-piece closure 180 that function together to provide the independent lock functionality. Moreover, it will be appreciated that the multiplanar receiver 100 and multiplanar cap retainer 150 of the IL multiplanar bone anchor assembly 26 shown in FIG. 66 can be the same as or substantially similar to those included in the multiplanar bone anchor assembly 20 described above, hence providing an additional degree of component-type modularity that can reduce the number of different individual components required to manufacture and assemble a spinal construct using the spinal fixation system 10 (see FIG. 1) of the present disclosure.

Illustrated in FIGS. 66-67 is the IL load saddle 290 that forms a component of the IL insert sub-assembly 204. The IL load saddle 290 can be substantially similar to the non-IL load saddle 230 described above, with the exception that the insert arms 292 are longer so that the top surfaces 291 of the insert arms 292 are located above the elongate rod.

With reference to FIGS. 67-72, the two-piece closure 180 can include an outer ring 190 comprising a generally cylindrical body 192 having an annular top surface 191, an annular bottom surface 193, and a central through-aperture 194. A continuous guide and advancement structure 196 with a start 197 can be formed into the side surfaces of the cylindrical body 192 and configured to rotatably mate with the discontinuous guide and advancement structure formed into interior faces of the upright arms of the receiver. As shown in the drawings, the guide and advancement structure 196 can be a helically-wound interlocking flange that is mateable with the complementary flange formed into the multiplanar receiver 100. Nevertheless, and as described above with reference to the receiver, other versions of the continuous guide and advancement structure 196 complementary with that formed into the multiplanar receiver are also possible and considered to fall within the scope of the present disclosure.

The central through-aperture 194 of the outer ring 190 includes an internal guide and advancement structure, in this case an internal thread 198, that is configured to threadably receive the center screw 182. As can be seen in the drawing figures, the center screw 182 also comprises a generally cylindrical body 184, but one that is much smaller and having an external or outer thread 188 that is complementary with the internal thread 198 of the central through aperture 194. The center screw 182 further includes an annular top surface 183, a solid or continuous bottom surface 185, and a central closed-off aperture 186 formed as a drive structure or internal drive socket 187 extending downwardly from the annular top surface 183 toward the bottom surface. The outer ring 190 can also have a drive structure, in this case a plurality of downward-extending recesses 195 formed into the upper portion of the central through-aperture 194, and which may interrupt the upper portions of the internal thread 198.

As discussed in more detail below, the annular bottom surface 193 of the outer ring 190 is configured to engage with the top surfaces 291 of the insert arms 292 of the IL pressure insert 290, while the closed-off bottom surface 185 of the center screw 182 is configured to engage the elongate rod. Other aspects of the two-piece closure 180 will be apparent to one of skill in the art upon further review of the drawing figures.

Illustrated in FIG. 73 is the multiplanar IL receiver sub-assembly 28 after the pre-assembly of the multiplanar cap retainer 150 and the IL insert sub-assembly 204 into the shipping state position within the receiver 100, in which the multiplanar cap retainer is 150 captured by the clamp positioner 250 in a position that is suspended and centered over the bottom opening 145 of the receiver 100.

Illustrated in FIG. 74 is the multiplanar IL bone anchor assembly 26 after the uploading of the shank head 60 through the bottom opening 145 of the receiver sub-assembly 28 and the downwardly deployment of the IL insert sub-assembly 204 (and the shank head sub-assembly 221) with the tool or tooling that can drive the lower ends of the clamp panels 260 of the clamp positioner 250 into engagement within the annular locking recess 142 of the receiver 100 and the upper outer rims 222 of the support collar 210 snap under the arcuate planar underside surfaces 134 of the opposed horizontal ridges 132 of the central bore 120. Upon completion of this downward deployment of the IL insert sub-assembly 204 within the receiver 100, the support collar 210 and the clamp positioner 250 can be rigidly securing as a unit to the internal structures of the central bore 120 and the pre-lock friction fit can be established between the shank head sub-assembly 21 and the discontinuous spherical inner surfaces 262 of the clamp positioner 250, due to the downwardly directed force created by the wave washer 170 being compressed between the bottom surfaces of the IL load saddle 290 and the upper surfaces of the crown member 280.

Illustrated in FIGS. 75-76 is the multiplanar IL bone anchor assembly 26 during final assembly with the elongate rod 4 and the two-piece closure 180. With initial reference to FIG. 75, the annular bottom surface 193 of the outer ring 190 of the two-piece closure 180 can engage the top surfaces 291 of the insert arms 295 of the IL load saddle 290, to drive the IL load saddle 290 downward until the annular bottom contact surface of the load saddle 190 directly engages the annular top contact surface of the crown member 180. Further torquing of the outer ring 190 of the two-piece closure 180 can then operate to hard lock the partial spherical outer surface of the cap retainer 150 against the discontinuous curvate inner surface of the clamp positioner 250 to prevent further movement of the shank 50 relative to the IL receiver sub-assembly 28. With continued reference to FIG. 76, the inner set screw 182 of the two piece closure 180 can then be threaded downward within the outer ring 190 until the bottom surface 185 of the inner ring 182 engages the elongate rod 4, to drive the elongate 4 rod downward into the insert channel 293 of the IL load saddle 290 and ultimate lock the elongate rod 4 within the multiplanar IL bone anchor assembly 26.

It will be appreciated that, similar to first multiplanar embodiment described above, subsequent limited unthreading or backing-off of the inner set screw and outer ring of the two-piece closure from the receiver, without removing the elongate rod or completely detaching the two-piece closure, can allow the wave washer to press the IL load saddle back upward within the rod channel to release the lock of the multiplanar IL bone anchor assembly 26 and re-establish the non-floppy, friction fit configuration between the components of the multiplanar IL bone anchor assembly 26. A slight wiggling of the multiplanar IL receiver sub-assembly 28 can then serve to re-mobilize the multiplanar IL receiver sub-assembly 28 relative to the bi-spheric shank head 60 and allow its position to be adjusted prior to re-locking the multiplanar IL bone anchor assembly 26 in a new position.

### Monoplanar Bone Anchor Assembly

Referring now to FIG. 77, illustrated therein is an exploded perspective view of one representative embodiment of the monoplanar bone anchor assembly 30 that is configured, as noted above, to limit the pivotal motion of the shank 50 relative to the monoplanar receiver sub-assembly 32 (or vice versa) to a single plane while still providing for a 360-degree range of rotational motion around the longitudinal axis of the shank 50. The monoplanar bone anchor assembly 30 can include the same bone anchor or shank 50 described above, having a bi-spherical universal shank head 60 and an anchor portion 84 opposite the bi-spheric shank head 60 for securement or attachment to the bone of a patient.

Similar to the multiplanar bone anchor assembly discussed above, the monoplanar bone anchor assembly 30 can include a monoplanar receiver 300 that can be initially pivotally secured to the bi-spherical universal shank head 60 with a number of separate internal components that have been pre-assembled into the central bore and the rod channel to form the monoplanar receiver sub-assembly 32. These internal components can include, but are not limited to, a pivoting or articulating monoplanar cap retainer 330 and a monoplanar insert sub-assembly 350. The monoplanar insert sub-assembly 350 can, in turn, be comprised a number of additional components, including but not limited to the support collar 210 that is coupled around the upper end of a monoplanar clamp positioner 370 and the lower end of the load saddle 230, with the additional wave washer 270 and crown element 280 being uploaded through the clamp positioner 360 and into the central aperture of the support collar 210 to complete the monoplanar insert sub-assembly 350. After an elongate rod has been positioned within the lower portion of the rod channel, the same single-piece closure (or any other appropriate type of closure) can be threadably or otherwise secured into an upper portion of the rod channel to apply pressure to an upper surface of the elongate rod, thereby locking both the elongate rod and the monoplanar bone anchor assembly 30 into a final or hard locked position.

Differences between monoplanar bone anchor assembly of FIG. 77 and the multiplanar bone anchor assembly previously described can include the replacement of the multiplanar receiver with a monoplanar receiver 300 (see FIGS. 78-79) having many of the same features, with the addition of opposed pairs of semi-circular upward-facing grooves and side pockets formed into the stepped seating structure and the sidewalls of the internal cavity, respectively. Additional differences can include the replacement of the multiplanar cap retainer with a monoplanar cap retainer 330 having opposite protrusions or pegs that project outwardly from opposite sides of the partial spherical outer surface (FIGS. 80-82), and the replacement of the multiplanar clamp positioner with a monoplanar clamp positioner 370 having opposed side apertures configured to receive the opposite pegs of the monoplanar cap retainer (FIGS. 83-84). As shown below, the opposite pegs are generally configured to be positioned into the semi-circular upward-facing grooves formed into the stepped seating structure, thereby defining the single pivot plane of the shank 50 relative to the monoplanar receiver 300.

It is foreseen that both the monoplanar receiver 300 and the components of the monoplanar insert sub-assembly 350 may also be configured differently in order to better interact with the opposite pegs of the monoplanar cap retainer 330. Nevertheless, the remainder of the components forming the monoplanar bone anchor assembly, such as the bi-spheric shank head 60, the elongate rod 4, and the closure 170, can be the same as or substantially similar to those already described, so as to provide the significant degree of component-type modularity that can reduce the number of different individual components required to manufacture and assemble a spinal construct using the spinal fixation system 10 (see FIG. 1) with all its attendant features and benefits, as discussed above.

Illustrated in FIGS. 85-86 is the pre-assembly of the monoplanar cap retainer 330 and insert sub-assembly 350 into the monoplanar receiver 300 to form the monoplanar receiver sub-assembly 32 in a shipping state position, in which the monoplanar cap retainer 330 is captured by the monoplanar clamp positioner 370 in a position that is suspended and centered over the bottom opening of the monoplanar receiver 300.

Illustrated in FIGS. 87-88 is one representative method of assembling the monoplanar receiver sub-assembly 32 to the bi-spheric shank head 60 of the shank 50, which can include the securement of the monoplanar cap retainer 330 around the upper partial spherical portion 64 of the bi-spheric shank head 60 to create an articulating monoplanar shank head sub-assembly 31 upon the uploading of the shank head 60 through the bottom opening, after which the insert sub-assembly 350 and the shank head sub-assembly 31 can be downwardly deployed with the tool or tooling until the discontinuous bottom edge portions of the monoplanar clamp positioner 370 become engaged within the annular locking recess of the monoplanar receiver 300. As with the multiplanar embodiments described above, this action can be simultaneous with the opposite integral upper flanges of the support collar 210 snaping under the arcuate underside surfaces of the opposed horizontal ridges of the monoplanar receiver 300, thereby rigidly securing the support collar 210 and the monoplanar clamp positioner 370 as a unit to the internal structures of the central bore and preventing these outer components of the monoplanar insert sub-assembly 350 from further movement within the monoplanar receiver 300.

Upon completion of this downward deployment of the monoplanar insert sub-assembly 350 within the monoplanar receiver 300, but prior to final assembly with the elongate rod and closure top, the articulating monoplanar shank head sub-assembly 31 (i.e. the monoplanar cap retainer 330 and the bi-spheric shank head 60) can be secured against the discontinuous curvate inner surface of the two-piece clamp positioner 370 with a frictional engagement, or pre-lock friction fit, that allows for the bone anchor or shank 50 to pivot relative to the monoplanar receiver 300 as the outer surfaces of the articulating monoplanar shank head sub-assembly 31 slidably frictionally engage with the discontinuous curvate inner surface of the monoplanar clamp positioner 370 at the same time that the opposite pegs pivot within the upward-facing grooves. This friction fit can be provided by the wave washer 270 pressing downward upon the crown element 280 that, in turn, applies the downwardly directed force to the partial spherical outer surface of the monoplanar cap retainer 330, thereby creating the frictional engagement between the articulating monoplanar cap retainer 330 and the fixed monoplanar clamp positioner 370.

Furthermore, while the engagement between the opposite pegs and opposed grooves can prevent the monoplanar clamp positioner 370 from rotating relative to the monoplanar receiver 300, the monoplanar receiver sub-assembly 32 may nevertheless be rotatable relative to the bi-spheric shank head by applying enough torque to overcome a frictional engagement at the minor diameter interface between the partial spherical inner surface of the monoplanar cap retainer 330 and the upper partial spherical portion of the bi-spheric shank head.

Illustrated in FIGS. 89-90 is the monoplanar bone anchor assembly 30 after final assembly with the elongate rod and the single piece closure, in which the load saddle 230 can be pressed downward by the elongate rod and closure until the annular bottom contact surface of the load saddle 230 directly engages the annular top contact surface of the crown member. Further tightening of the closure to a predetermined level of torque can serve to lock the partial spherical outer surface and the opposite pegs of the monoplanar cap retainer 330 against the discontinuous curvate inner surface of the monoplanar clamp positioner 370 and the upward-facing grooves of the stepped seating structure of the monoplanar receiver 300, respectively, thereby preventing further movement of the shank relative to the monoplanar receiver 300 and fully locking the monoplanar bone anchor assembly 30 together with the elongate rod.

It will be appreciated that, similar to the multiplanar embodiments described above, subsequent limited unthreading or backing-off of the closure from the monoplanar receiver can allow the wave washer 270 to press the load saddle 230 back upward within the rod channel and thereby release the internal lock of the assembly. A slight wiggling of the monoplanar receiver sub-assembly 32 can then serve to re-mobilize the monoplanar receiver 300 sub-assembly back into the non-floppy, friction fit configuration with the bi-spheric shank head 60, so that its orientation and/or the position of the elongate rod may be adjusted prior to re-locking the monoplanar bone anchor assembly 30 in a new position.

### Monoaxial Bone Anchor Assembly

Referring now to FIG. 91, illustrated therein is an exploded perspective view of one representative embodiment of the monoaxial or ‘non-pivotal but rotatable’ bone anchor assembly 40 that is configured to substantially eliminate pivotal motion of the bone anchor relative to the receiver sub-assembly, except perhaps for a slight toggle, while still providing for a 360-degree range of rotational motion around the longitudinal axis of the bone anchor. The monoaxial bone anchor assembly 40 can include the same bone anchor or shank 50 described above, having a bi-spherical universal shank head 60 and an anchor portion 84 opposite the bi-spheric shank head 60 for securement or attachment to the bone of a patient.

Related to the multiplanar and monoplanar bone anchor assemblies discussed above, the monoaxial bone anchor assembly 40 generally includes the receiver 100 that can be rotatably secured to the bi-spherical universal shank head 60 with a number of separate internal components that have been pre-assembled into the central bore and the rod channel to form the monoaxial receiver sub-assembly 42. These internal components can include, but are not limited to, a monoaxial cap retainer 430 and a monoaxial insert sub-assembly 450. The monoaxial insert sub-assembly 450 can, in turn, comprise a number of additional components including the central collar, a monoplanar load saddle 460, and the two-piece clamp positioner 250, with the support collar 210 being coupled around both the upper end of the clamp positioner 250 and the lower end of the monoplanar load saddle 460. The monoaxial insert sub-assembly 450 can also include the wave washer 270 that can initially be placed on the lower stepped surface of an upper stepped ring portion of the monoaxial cap retainer 430, for uploading with the monoaxial cap retainer 430 through the lower opening of the clamp positioner 250 and into the central aperture of the central collar. After an elongate rod has been positioned within the lower portion of the rod channel, the same single-piece closure (or any other appropriate type of closure) can be threadably or otherwise secured into an upper portion of the rod channel to apply pressure to an upper surface of the elongate rod, thereby locking both the elongate rod and the monoaxial bone anchor assembly into a final locked position.

The receiver 100 of the monoaxial bone anchor assembly illustrated in FIGS. 92-93 can have substantially the same construction and features of the receiver of the multiplanar bone anchor assembly shown in FIGS. 7-10, and in one aspect the two embodiments of the receiver can be interchangeable or even identical. This is possible because the differences between the multiplanar and monoaxial bone anchor assemblies can be limited to the interface between the upper portions of the cap retainers and the lower internal portions of the insert sub-assemblies. Thus, regarding the lower portions of the multi-piece clamp positioner 250 (i.e. the discontinuous bottom edge portion), the outer portions of the monoaxial load saddle (i.e. the opposite indexing nubs), and outer portions of the support collar 210 (i.e. the opposite upper outer flanges), these structures and/or surfaces of the monoaxial embodiment can interact with the internal structures and/or surfaces of the central bore of the receiver 100 in substantially the same way as those of the multiplanar embodiment.

Differences between the multiplanar embodiments previously described and the monoaxial bone anchor assembly of FIG. 91 can include the replacement of the multiplanar cap retainer with a non-pivotal monoaxial cap retainer 430 and the multiplanar load saddle with a monoaxial load saddle 460. As shown in FIGS. 94-96, for example, the monoaxial cap retainer 430 can include a stepped upper ring portion having a raised center cylinder with a top annular surface defining an upper central opening. In turn, the raised center cylinder can be surrounded by a lower stepped surface that extends radially outward to a lower cylindrical outer surface. The monoaxial cap retainer 430 can further include a partial spherical lower outer surface defined by the resilient collet fingers, and which extends upwardly from the discontinuous bottom surface and then transitions upwardly into the lower cylindrical outer surface of the stepped upper ring portion.

Illustrated in FIG. 97 is an exploded perspective view of the monoaxial cap retainer 430 and the monoaxial insert sub-assembly 450 in isolation.

With reference to FIGS. 98-100, the bottom portions of the monoaxial insert sub-assembly 450 can also be modified, with the crown element being removed and the center opening of the monoaxial load saddle 460 being expanded so as to slidingly receive the center cylinder of the stepped upper ring portion of the monoaxial cap retainer 430. In addition, the opposite lower flanges of the monoaxial load saddle 460 that engage with interior surfaces of the support collar 210 can be provided with downward-extending lips that serve to maintain the wave washer 270 in position between the bottom annular surface of the monoaxial load saddle 460 and the lower stepped surface of the monoaxial cap retainer 430, upon the assembly of the monoaxial cap retainer 430 together with the monoaxial insert sub-assembly 450 into the shipping state position within the receiver 100.

Illustrated in FIGS. 101-107 is the pre-assembly of the monoaxial cap retainer 430 and monoaxial insert sub-assembly 450 into the receiver 100 to form the monoaxial receiver sub-assembly 42 in the shipping state position, in which the monoaxial cap retainer 430 is captured by the clamp positioner 250 in a position that is suspended and centered over the bottom opening of the receiver 100.

Illustrated in FIG. 108 is the monoaxial receiver sub-assembly 42 after the uploading of the bi-spheric shank head 60 of the shank 50 through the bottom opening of the receiver 100 and into the monoaxial cap retainer 430, after which the monoaxial insert and shank head sub-assemblies can be downwardly deployed until the discontinuous bottom edge portions of the clamp positioner 250 become engaged within the annular locking recess of the receiver 100. As with the previous embodiments described above, this action can be simultaneous with the opposite integral upper flanges of the support collar 210 snaping under the arcuate underside surfaces of the opposed horizontal ridges, thereby rigidly securing the support collar 210 and the clamp positioner 250 as a unit to the internal structures of the central bore and preventing these outer components of the monoaxial insert sub-assembly 450 from further movement within the receiver 100.

Similar to the earlier embodiments, upon the initial assembly of the monoaxial receiver sub-assembly 42 to the bi-spheric shank head 60, but prior to final assembly with the elongate rod and closure top, the non-pivoting but rotatable monoaxial shank head sub-assembly (i.e. the monoaxial cap retainer 430 and the bi-spheric shank head 60) can be rotatably secured within the two-piece clamp positioner 250 with a frictional engagement, or pre-lock friction fit. In particular, the bone anchor or shank 50 may be rotated relative to the receiver 100 as the partial spherical lower outer surface of the monoaxial cap retainer 430 slidably frictionally engages with the discontinuous curvate inner surface of the clamp positioner 250. In one aspect this friction fit can be provided by the wave washer 270 pressing directly downward upon the stepped upper ring portion of the monoaxial cap retainer 430, thereby creating the frictional engagement between the outer surfaces of the monoaxial cap retainer 430 and the inner surfaces of the clamp positioner 250.

It will be appreciated that the sliding cylindrical engagement between the lower cylindrical outer surface of the stepped upper ring portion of the monoaxial cap retainer 430 and the cylindrical inner surfaces of the upper connection portion of the clamp positioner 250 can operate to prevent the monoaxial cap retainer 430, and thus the bi-spheric shank head 60 that is non-pivotably coupled to the monoaxial cap retainer 430, from pivoting relative to the receiver 100. In one aspect the sliding cylindrical connection between the raised center cylinder of the monoaxial cap retainer 430 and the center opening of the monoaxial load saddle 460, as shown in FIGS. 109 and 110, can also contribute to the prevention of the monoaxial cap retainer 430 from pivoting relative to the receiver 100.

Illustrated in FIG. 109 is the monoaxial bone anchor assembly 40 after final assembly with the elongate rod and the single piece closure, in which the elongate rod and/or the fully compressed wave washer 270 can be pressed downward against the monoaxial cap retainer 430 by the closure. In this configuration the elongate rod may directly engage the top annular surface and/or the fully compressed wave washer 270 may engage the lower stepped surface to lock the partial spherical lower outer surface of the monoaxial cap retainer 430 against the discontinuous curvate inner surface of the clamp positioner 250, thereby preventing further rotational movement of the shank relative to the receiver 100 and fully locking the monoaxial bone anchor assembly 40 together with the elongate rod.

As with the previously-described embodiments, it will be appreciated that subsequent limited unthreading or backing-off of the closure from the receiver 100 can allow the wave washer 270 to press the monoaxial load saddle 460 back upward within the rod channel and thereby release the internal lock of the assembly. A slight wiggling or twisting of the monoaxial receiver sub-assembly 42 can then serve to re-mobilize the monoaxial receiver sub-assembly 42 back into the friction fit configuration with the bi-spheric shank head 60, so that its rotational angular position or that of the elongate rod may be adjusted prior to re-locking the monoaxial bone anchor assembly 40 in a new position.

### Semi-Fixed Monoaxial Bone Anchor Assembly

Referring now to FIG. 110, illustrated therein is an exploded perspective view of another representative embodiment of the monoaxial bone anchor assembly 46 that, similar to the first embodiment of the monoaxial bone anchor assembly discussed above, is configured to substantially eliminate pivotal motion of the receiver sub-assembly relative to the bone anchor, except perhaps for a slight toggle. In contrast to the previous embodiment, however, the monoaxial bone anchor assembly 46 can be configured to provide a more restricted or limited rotational motion of the receiver sub-assembly 48 about the longitudinal axis of the shank 50 due to an increased level of frictional interference between the internal components and/or surfaces of the receiver sub-assembly 48. Accordingly, in one aspect the monoaxial bone anchor assembly of FIG. 110 may also be considered a “semi-fixed” assembly that substantially eliminates pivotal motion of the receiver sub-assembly 48 relative to the shank 50 while allowing for restricted, limited, or high-frictional rotational motion of the receiver sub-assembly 48 about the longitudinal axis of the shank 50.

The receiver 100 of the monoaxial or semi-fixed bone anchor assembly illustrated in FIGS. 111-112 can have substantially the same construction and features of the receivers of both the multiplanar bone anchor assembly shown in FIGS. 7-10 and the monoaxial bone anchor assembly shown in FIGS. 92-93, and in one aspect the embodiments of the receiver can be interchangeable or even identical. As with the previous embodiments, the semi-fixed monoaxial receiver sub-assembly 46 can be non-pivotally secured to the bi-spherical universal shank head 60 with a number of internal components that have been pre-assembled into the central bore and rod channel to form the monoaxial or semi-fixed receiver sub-assembly 48. These internal components can include, but are not limited to, the monoaxial insert sub-assembly 450 described above and an alternative or semi-fixed embodiment of the monoaxial cap retainer 480 having a plurality of laterally-outward-extending partial cylindrical stubs or pegs.

With reference back to FIG. 110, the monoaxial insert sub-assembly 450 can be substantially similar or even identical to the monoaxial insert sub-assembly 450 described above, comprising the support collar 210, the monoaxial load saddle 460, the two-piece clamp positioner 250, and the wave washer 270. With the absence of the crown element, the wave washer 270 that can initially be placed on the lower stepped surface of the stepped upper ring portion of the semi-fixed monoaxial cap retainer 480 prior to uploading the cap retainer into the insert sub-assembly 450 through the lower opening of the clamp positioner 250 and into the central aperture of the central collar. After an elongate rod has been positioned within the lower portion of the rod channel, the same single-piece closure (or any other appropriate type of closure) can be threadably or otherwise secured into an upper portion of the rod channel to apply pressure to an upper surface of the elongate rod, thereby locking both the elongate rod and the semi-fixed monoaxial bone anchor assembly 46 into a final or hard locked position.

Differences between the first monoaxial bone anchor assembly 40 of FIG. 91 and the semi-fixed monoaxial bone anchor assembly 46 of FIG. 110 can include the addition of the plurality of stubs or pegs (in this case, four equally-spaced stubs having a partial cylindrical shape) extending laterally outward from the stepped upper ring portion of the semi-fixed monoaxial cap retainer 480, as shown in FIGS. 113-115. Other aspect of the semi-fixed monoaxial cap retainer 480 can be substantially similar or even identical with the first monoaxial cap retainer 430 described above, including but not limited to the stepped upper ring portion having the raised center cylinder with the upper central opening defining the top annular surface, and which is surrounded by the lower stepped surface that extends radially outward to a lower cylindrical outer surface. The semi-fixed monoaxial cap retainer 480 can also include the partial spherical lower outer surface defined by the resilient collet fingers that extends upwardly from the discontinuous bottom surface, and then transitions upwardly into the lower cylindrical outer surface of the stepped upper ring portion.

During the assembly of the pre-assembly of the semi-fixed monoaxial cap retainer 480 and monoaxial insert sub-assembly 450 with the receiver to form the semi-fixed monoaxial receiver sub-assembly 48 in the shipping state position, as shown in FIGS. 116-123, the laterally-outward-extending stubs can be slidably received within the vertical slots formed into the lower expandable portions of the clamp positioner 250 as the semi-fixed monoaxial cap retainer 480 is uploaded through the lower opening of the clamp positioner 250 and into the central aperture of the support collar 210 and the center opening of the monoaxial load saddle. The stubs can operate to rotationally lock the semi-fixed monoaxial cap retainer 480 to clamp positioner 250 so that the two components either rotate together or become fixed together after the uploading of the bi-spheric shank head 60 into the receiver sub-assembly 48, as shown below. With reference to the shipping state configuration shown in FIG. 123, the semi-fixed monoaxial cap retainer 480 is captured by the clamp positioner 250 in a position that is suspended and centered over the bottom opening of the receiver.

Illustrated in FIG. 124 is the semi-fixed monoaxial receiver sub-assembly 48 after the uploading of the bi-spheric shank head 60 of the shank 50 through the bottom opening of the receiver and into the semi-fixed monoaxial cap retainer 480, after which the monoaxial insert and shank head sub-assemblies can be downwardly deployed until the discontinuous bottom edge portions of the clamp positioner 250 become engaged within the annular locking recess of the receiver. As with the previous embodiments described above, this action can be simultaneous with the opposite integral upper flanges of the support collar 210 snaping under the arcuate underside surfaces of the opposed horizontal ridges, thereby rigidly securing the support collar 210 and the clamp positioner 250 as a unit to the internal structures of the central bore and preventing these outer components of the monoplanar insert sub-assembly 450 from further movement within the receiver.

With the semi-fixed monoaxial cap retainer 480 being rotationally locked to clamp positioner 250, and the clamp positioner, in turn, being rotationally locked to the internal surfaces of the receiver, the rotation of the semi-fixed receiver sub-assembly 48 about the longitudinal axis of the shank 50 can be restricted or limited to the frictional engagement at the minor diameter interface between the upper partial spherical portion of the bi-spheric shank head 60 and the discontinuous inner spherical surface of the semi-fixed monoaxial cap retainer 480, thereby provided for the increased frictional engagement described above.

Illustrated in FIG. 125 is the semi-fixed bone anchor assembly 46 after final assembly with the elongate rod and the single piece closure, in which the elongate rod and/or the fully compressed wave washer 270 can be pressed downward against the semi-fixed monoaxial cap retainer 480 by the closure. In this configuration the elongate rod may directly engage the top annular surface and/or the fully compressed wave washer 270 may engage the lower stepped surface to lock the partial spherical lower outer surface of the semi-fixed monoaxial cap retainer 480 against the discontinuous curvate inner surface of the clamp positioner 250, while also strongly clamping the plurality of flexible collet fingers about the upper partial spherical portion of the bi-spheric shank head 60, thereby preventing further rotational movement of the shank relative to the receiver and fully locking the semi-fixed bone anchor assembly 46 together with the elongate rod.

As with the previously-described embodiments, it will be appreciated that subsequent limited unthreading or backing-off of the closure from the receiver can allow the wave washer 270 to press the monoaxial load saddle back upward within the rod channel and thereby release the internal lock of the assembly. A slight wiggling or twisting of the semi-fixed receiver sub-assembly 48 can then serve to re-mobilize the receiver sub-assembly back into the friction fit configuration with the bi-spheric shank head 60, so that its rotational angular position or that of the elongate rod may be adjusted prior to re-locking the semi-fixed bone anchor assembly 46 in a new position.

### Closed Support Collar

Referring now to FIG. 126, illustrated therein is an exploded perspective view of another representative embodiment of the multiplanar bone anchor assembly 24 and multiplanar receiver sub-assembly 25 that, similar to the first embodiment of the multiplanar bone anchor assembly discussed above, are configured to provide for continuous pivotal motion and rotation of the bone anchor 50 relative to the receiver sub-assembly 25 around a 360-degree range, and also to provide for pre-lock frictional axial rotation relative to a longitudinal axis of the bone anchor around a 360-degree range.

In contrast to the previous embodiment, however, the receiver sub-assembly 25 includes an insert sub-assembly 500 having a support collar 510 with ring-shaped body 212 that is closed, as shown in FIGS. 127-128. In one aspect the closed ring-shaped body can provide additional strength and stability to the insert sub-assembly 500 during deployment and use. In addition, it will be appreciated that the support collar 510 with the close ring-shaped body 212 may also be used with the monoplanar and monoaxial embodiments described above.

In providing the support collar 510 with the closed ring-shaped body 212, it may be necessary to modify the load saddle 530 and/or the clamp positioner 550 to facilitate their pre-assembly together (FIG. 131) without the flexing open of the support collar 510. For instance, in one aspect the planar end surfaces 555 of the upper connection portions 554 of the clamp positioner 550 can be provided with beveled or chamfered upper portions 561 to facilitate their uploading into the closed ring-shaped body 212, as shown in FIG. 129-130. Additional modifications to the alignment bar of the load saddle 210 and/or the upper inner rim of the support collar, as well as modification to other features of the various components, are also possible and considered to fall within the scope of the present disclosure.

Illustrated in FIG. 132 is the multiplanar receiver sub-assembly 25 after the uploading of the bi-spheric shank head 60 of the shank 50 through the bottom opening of the receiver 100 and into the cap retainer 550, after which the insert sub-assembly 500 and the shank head sub-assembly 21 can be downwardly deployed until the discontinuous bottom edge portions of the clamp positioner 250 become engaged within the annular locking recess of the receiver 100 as the opposite integral upper flanges of the support collar 510 snap under the arcuate underside surfaces of the opposed horizontal ridges.

As indicated above, the spinal fixation system of the present disclosure has been described herein in terms of preferred embodiments and methodologies considered by the inventors to represent best modes of carrying out the one or more inventions disclosed herein. It will be understood by the skilled artisan, however, that a wide range of additions, deletions, and modifications, both subtle and gross, may be made to the illustrated embodiments of the pivotal and non-pivotal bone anchor assemblies, to the modular spinal fixation system, and to the representative type of bi-spherical universal shank head, and that these and other revisions might be made by those of skill in the art without departing from the spirit and scope of the one or more inventions that are to be constrained only by their respective claims.

What is claimed is:

1. A universal shank head system for securing a fixation rod to a bone of a patient with closures, the universal shank head system comprising:
a plurality of bone anchors, each bone anchor comprising a longitudinal axis, a shank head at a proximal end, and an anchor portion at a distal end configured for fixation to the bone, the shank head including a planar top surface at an upper end of the shank head, an upper partial spherical surface having a first diameter extending downward from the planar top surface past a hemisphere plane to a circular inner edge of an upward-facing ledge, and a lower partial spherical portion having a second diameter greater than the first diameter extending downward from a discontinuous circular outer edge of the upward-facing ledge to a neck portion that connects the shank head to the anchor portion; and
a plurality of pivoting and non-pivoting receiver sub-assemblies, each receiver sub-assembly including:
a receiver having a vertical centerline axis, an upper portion defining a channel configured to receive the fixation rod, and a base defining a lower portion of a central bore formed around the vertical centerline axis and communicating with a bottom surface of the receiver through a bottom opening, the central bore extending upwardly from the bottom opening through the rod channel to a top of the receiver and including a guide and advancement structure mateable with a closure proximate the top of the receiver and a seating surface adjacent the bottom opening;
an insert sub-assembly positionable within the central bore below the guide and advancement structure, the insert sub-assembly comprising:
a support collar having outer surfaces configured to engage interior surfaces of the central bore and inner surfaces defining a central aperture;
a load saddle having an upper surface configured to engage the elongate rod and a pair of opposite, radially-projecting lower flanges configured to engage with inner surfaces of the central aperture of the central collar; and
a multi-piece clamp positioner having upper lip portions with outer surfaces configured to engage with inner surfaces of the central aperture of the support collar below the lower flanges of the load saddle, a discontinuous bottom edge portion configured to engage with the seating surface of the central bore of the receiver, and a discontinuous curvate inner surface; and
a retaining structure positionable within the internal cavity having a partial spherical outer surface configured to slidably engage the discontinuous curvate inner surface of the clamp positioner, a partial spherical inner surface configured to frictionally engage the upper partial spherical surface of the shank head, and a plurality of slots extending upward from a discontinuous annular bottom surface to form a plurality of flexible collet fingers configured to expand to receive the shank head when the shank head is uploaded through the bottom opening; and
wherein each of the shank heads is configured for uploading into both the pivoting and non-pivoting receiver sub-assemblies through the bottom opening of the receiver, and for axial rotation about the longitudinal axis of the shank relative to the receiver prior to locking the receiver sub-assembly to the head of the shank with the fixation rod and a closure.

2. The universal shank head system of claim 1, wherein the bone anchor further comprises a shank having an internal drive structure extending inward from the top surface and configured to mate with a drive tool.

3. The universal shank head system of claim 2, wherein the shank includes an axial bore extending from the internal drive structure down to a bottom end of the anchor portion and configured to receive a guide wire, the anchor portion of the shank being configured for implantation in the bone about a guide wire with the drive tool prior to the shank head being uploaded into the central bore of the receiver through the bottom opening.

4. The universal shank head system of claim 1, wherein the upward-facing ledge and a lower partial spherical portion of the shank head further comprise a plurality of open, vertically aligned flutes arranged circumferentially around the shank head and extending downwardly through and below the upward-facing ledge.

5. The universal shank head system of claim 1, further comprising a discontinuous upper recess formed into the central bore of the receiver and a discontinuous downward-facing lower arcuate surface below discontinuous upper recess,
wherein opposite integral upper flanges projecting radially outward from upper portions of the support collar with top surfaces are configured to rotate into the discontinuous upper recess formed into the central bore of the receiver, and
wherein the support collar is downwardly deployable within the central bore until the opposite integral upper flanges snap under the discontinuous downward-facing lower arcuate surface to prevent the support collar and the clamp positioner of the insert sub-assembly from moving back up within the central bore of the receiver.

6. The universal shank head system of claim 1, wherein the insert sub-assembly further comprises wave washer positionable within the central aperture of the support collar and engageable with an annular bottom surface of the load saddle, the wave washer being operable to apply a downwardly directed force to the retaining structure to establish a frictional engagement between the retaining structure and the clamp positioner to inhibit motion therebetween.

7. The universal shank head system of claim 6, wherein the insert sub-assembly further comprises crown element positionable within the central aperture of the support collar having a stepped upper surface configured to engage the wave washer and a partial spherical downward-facing surface configure to apply the downwardly directed force to the partial spherical outer surface of the retaining structure to establish the frictional engagement between the retaining structure and the clamp positioner.

8. The universal shank head system of claim 6, wherein the retaining structure further comprises a stepped upper ring portion having a raised center cylinder configured for slidable engagement with a center opening of the load saddle and a lower stepped surface configured to receive the downwardly directed force from the wave washer to establish the frictional engagement between the retaining structure and the clamp positioner.

9. The universal shank head system of claim 1, wherein the plurality of pivoting receiver sub-assemblies further comprises at least one multiplanar receiver sub-assembly and at least one monoplanar receiver sub-assembly.
